(19) United States
(12) Patent Application Publication
(10) Pub. No.: US 2025/0177590 A1
(43) Pub. Date: Jun. 5, 2025

(54) On-Demand Hypochlorous Acid (HOCL) Generator and Sprayer

(71) Applicant: MARSIX SOLUTIONS LTD., Winnipeg (CA)

(72) Inventors: Gregory Michael Mardero, Winnipeg (CA); Mitchell Stephan Mardero, Winnipeg (CA); Steve Gianpiero Mardero, Winnipeg (CA)

(21) Appl. No.: 18/842,263

(22) PCT Filed: Mar. 1, 2023
(86) PCT No.: PCT/CA2023/050264

§ 371 Date: Aug. 28, 2024

Related U.S. Application Data
(60) Provisional application No. 63/315,715, filed on Mar. 2, 2022.

(51) Int. Cl. A61L 2/22; A61L 2/24; A61L 101/06; C25B 1/26; C25B 9/17; C25B 9/60; C25B 15/025; C25B 15/08

(52) U.S. Cl. CPC A61L 2/22; A61L 2/24; C25B 1/26; C25B 9/17; C25B 9/60; C25B 15/025; C25B 15/08; A61L 2101/06; A61L 2202/14; A61L 2202/15; A61L 2202/16

(57) ABSTRACT

The present invention is a portable, on-demand hypochlorous acid (HOCL) generator and sprayer device contained in a wireless electrostatic backpack system designed for the purpose of sanitizing and disinfecting. The system converts a feeder solution of water mixed with sodium chloride acetic acid into a hypochlorous acid solution at adjustable concentrations (25-400 ppm) by electrolysis using both anode and cathode electrodes. The resulting solution is pressurized to 50-130 psi, and then atomized into an ultra-fine mist (40-80 microns) that passes through an electrically charged annular element to positively charge the atomized droplets, increasing the transfer efficiency to negative and neutrally charged surfaces by providing a wraparound, attractive force on surfaces sprayed. Various sensors, controls and a central processing unit (logic controller) are used to ensure the consistency and accuracy of the required output. The hypochlorous acid produced through this on-demand system is non-toxic and environmentally friendly.

This application claims priority benefits to U.S. Provisional Patent Application No. 63/315,715, filed Mar. 2, 2022.

### FIELD OF THE INVENTION

The present invention relates to sanitizer and disinfectant-producing devices, specifically devices that produce on-demand sprayable solutions using saline with electricity and electrolyzing cells in portable configurations.

### BACKGROUND

Portable battery-powered electrostatic sprayers in a backpack and hand-held versions are a rapidly growing industry used for sanitizing and disinfecting surfaces. These sprayers are typically electrostatic to aid in a full coverage and provide wrap-around surface contact during spraying. However, currently available devices have a significant drawback as they require the purchase of expensive consumable disinfectants or sanitizer products that are often toxic and unfriendly to the environment. These products are typically sodium hypochlorite, ammonia, or alcohol-based and require the user to wear protective equipment such as respirators and masks to minimize the risk of inhalation, chemical burns, and eye irritation.

Although hypochlorous acid can be purchased as a liquid disinfectant or sanitizer that is non-toxic and environmentally friendly, it is costly. It typically has a limited shelf life that loses efficacy quickly over time.

The use of electrolysis systems for hypochlorous acid has been developed for large establishments or the commercial sale of the generated solution. Generally, they are bulky, stationary devices. While there are hand-held spray bottles of hypochlorous solutions batch processed, they are not produced on demand for the user, nor are they consistently reliable, considering they have a limited shelf life.

In the electrolysis process, chlorine ions are oxidized at the anode to form chlorine when they combine with water to make hypochlorous acid. The PH level of the fluid determines the purity of the hypochlorous, where a PH of 4 to 6.5 is predominantly hypochlorous and a PH above 7 is predominately hypochlorite.

Therefore, a need exists in the field for a novel portable, on-demand hypochlorous acid-generating device and sprayer contained in a wireless electrostatic backpack system designed for sanitizing and disinfecting surfaces.

In addition, a further need is to have a hypochlorous producing device that can adjust the concentration of hypochlorous acid on demand in a range from 25 to 400 ppm or more that can meet requirements for 1) food contact at 60 ppm, with no rinsing required before use. 2) food contact surfaces at 100-200 ppm, 3) general surfaces not in contact with foods at 200+ ppm.

### SUMMARY OF THE INVENTION

According to one aspect of the invention there is provided a portable cleaning solution generating device comprising:
a supply tank arranged to contain a feeder solution;
an electrolytic cell comprising (i) a chamber, (ii) an inlet in communication between the chamber and the supply tank so as to be arranged to receive the feeder solution through the inlet into the chamber from the supply tank, (iii) two or more electrodes supported within the chamber so as to be arranged to produce the cleaning solution from the feeder solution by electrolysis, and (iv) a liquid outlet arranged to discharge the cleaning solution from the chamber through the liquid outlet;
a pump in communication with the liquid outlet of the electrolytic cell to produce a pressurized flow of the cleaning solution;
an atomizing nozzle in communication with the pump so as to be arranged to receive the pressurized flow of the cleaning solution and discharge the pressurized flow of the cleaning solution as an atomized mist; and
a controller arranged to actuate the electrodes of the electrolytic cell to produce the cleaning solution in the chamber when the pump is actuated to discharge the atomized mist from the atomizing nozzle.

In a preferred embodiment, the feeder solution comprises a mixture of water, sodium chloride and acetic acid therein, and the electrolytic cell is arranged to produce the cleaning solution from the feeder solution by electrolysis such that the cleaning solution comprises a hypochlorous acid solution.

In an alternative embodiment, the feeder solution comprises a mixture of water and potassium carbonate therein, and the electrolytic cell is arranged to produce the cleaning solution from the feeder solution by electrolysis such that the cleaning solution comprises a degreasing solution.

According to a further aspect of the present invention there is provided a portable hypochlorous acid generating device comprising:
a supply tank arranged to contain a feeder solution comprising a mixture of water, sodium chloride and acetic acid therein;
an electrolytic cell comprising (i) a chamber, (ii) an inlet in communication between the chamber and the supply tank so as to be arranged to receive the feeder solution through the inlet into the chamber from the supply tank. (iii) two or more electrodes supported within the chamber so as to be arranged to produce a hypochlorous acid solution from the feeder solution by electrolysis, and (iv) a liquid outlet arranged to discharge the hypochlorous acid solution from the chamber through the liquid outlet;
a pump in communication with the liquid outlet of the electrolytic cell to produce a pressurized flow of the hypochlorous acid solution;
an atomizing nozzle in communication with the pump so as to be arranged to receive the pressurized flow of the hypochlorous acid solution and discharge the pressurized flow of the hypochlorous acid solution as an atomized mist; and
a controller arranged to actuate the electrodes of the electrolytic cell to produce the hypochlorous acid solution in the chamber when the pump is actuated to discharge the atomized mist from the atomizing nozzle.

The present invention enables hypochlorous acid to be produced on demand in a battery-powered backpack configuration. The conversion process uses an acidic saline solution and applies electricity passing between a cathode and anode electrode. The hypochlorous solution is then pressurized and flows into the spray gun and out the atomizing nozzle. As the spray mist exits the nozzle, an electrostatic charge is imparted to the mist droplets. The electrostatically positive charge on the mist is attracted to the negative or neutral-charged surfaces being sprayed. improving the transfer efficiency of the spraying function. The electrolysis process enables the production of hypochlorous acid to various concentrations required for immediate application to surfaces.

One embodiment incorporates a backpack strap to allow users to wear the device on their backs. A retractable handle and wheel arrangement also allow the device to be rolled around for transportation and can also be used for spraying as its being wheeled.

In the preferred embodiments, the devices comprises: a feeder mixture solution that gravity feeds down from the tank using a fluid consisting of 99.3% water mixed with 0.2% Salt (Sodium Chloride) and 0.5% Vinegar (Acidic acid); a quick connect coupler to provide for one step tank removal for refilling; an electrolysis cell and pump where current is applied across an anode and cathode to convert the mixture into a hypochlorous acid in 25-400 ppm range; an automatic gas release float valve to safely displace hydrogen gases produced during the conversion while preventing any liquid from escaping; a 12 volt LiFePo4 battery; a fluid pump that pressurizes the liquid; a spray gun that contain an misting nozzle that creates a ultra-fine mist; an annular conductive ring that transfers electrostatic charges to the mist to provide attractive force that wraps around on surfaces: a touch screen display for user interface for system settings and to provide error messaging and instructions to the user, sensors for PH and salinity feedback and logic controller to control and monitor the process to ensure quality and consistency of the conversion.

The rectangular anode and cathode are contained in a PVC or similar corrosion-resistant material and are supplied with a feeder solution from the tank that enters the top of the conversion cell that is mounted vertically. As the liquid flows downward, fluid swirls and mixes through the baffle arms, further directing the flow to the center of the electrodes. The baffle arms are part of a one-piece baffle insulator mount that separates the two electrodes by approx. 0.04″ for optimum operation. The flow then exits the bottom of the cell while repeatedly mixing and flowing through the electrodes on its pathway out as it navigates through the baffle arms.

The baffle insulator and arms are further designed to direct the created hydrogen gasses upwards and directly to the center of the cell to the top-centered gas outlet port. The cell is directly mounted and connected to the pressurizing pump via two standoff mounts. This allows the vibration created by the pump and motor combination to transfer the harmonic frequency to the electrodes. The harmonic frequency assists in displacing any gas bubbles that build up on the electrode surfaces or chamber walls, causing them to break away and flow out of the gas exit port, increasing conversion efficiency.

The electrolysis cell also incorporates a clear PVC cover that allows the user to view the conversion process providing real-time visualization of the function occurring. LED lighting is also incorporated into a clear cover to improve the visibility of the process.

The gasses that flow out the gas exit port flow into an automatic gas release valve. The valve consists of a float bowl and float that activates a sealing element that seals off the flow when the fluid level is high, preventing fluid from passing. When the float level fills with gasses, the float lowers, releasing the gasses without any fluids being released. The gas is safely displaced into the ambient air or alternatively through a small diameter tube.

The tank is connected to the backpack system with a drop-in quick-connect coupler made of PVC or similar corrosion-resistant material. The couplers are normally closed check valves with one prong on the tank and the other a socket-style receiver mounted to the tank holder. The normally closed check valves prevent leakage of fluids when the tank is removed for filling. Once the tank is positioned in the tank holder, the normally closed check valves are mechanically activated and opened to allow the free flow of fluid to the electrolysis cell.

When the spray gun is activated, the fluid pump pressurizes the hypochlorous fluid mixture, and the fluid flows to the spray gun and out the atomizing nozzle creating an ultrafine mist of 40 to 80 microns. The spray then passes through a conductive annular ring that transfers an electrostatic charge of 2-20 kv to the spray droplets. The positively charged droplets are attracted to the negative or neutral charged surfaces, significantly increasing the transfer efficiency and creating a warp-around effect. The high-voltage transformer that supplies the voltage to the annular ring is contained in the spray gun directly in front of the fan. The fan is incorporated into the back of the spray gun to provide an airflow assist to the spray pattern, increasing the range and coverage of the spray.

A touch display screen and logic controller are used to set parameters for the conversion process with various settings of hypochlorous 60 ppm for food contact, 100-200 for food contact surfaces, and 200+ for general surfaces replacing the need for three different sanitizers or disinfectants. (Other concentrations are also possible) The control system has sensors for voltage, current, and fluid tank levels. During the conversion process, the system monitors the voltage and current. It maintains a set total power to the electrodes to provide a consistent hypochlorous concentration based on the set point. The power output is maintained over a range of salt content and resulting electrical conductivity of the fluid. The process monitoring also includes upper and lower thresholds that automatically shut down the device when the power is out of range, ensuring accurate control of the concentration. The process monitoring also includes a low and high PH threshold with a PH sensor in contact with the feeder solution that shuts down the device when it is not in range.

According to any embodiments described above, the device may further include a trigger switch operatively connected to the controller, the controller being arranged to operate the pump and the electrodes of the electrolytic cell together in response to actuation of the trigger switch.

According to any embodiments described above, the device may further include a user input in communication with the controller so as to be arranged to receive a selected hypochlorous acid concentration input from a user, wherein the controller is arranged to adjust voltage applied to the electrodes of the electrolytic cell in response the hypochlorous acid concentration input received by the controller whereby a concentration of hypochlorous acid within the hypochlorous acid solution can be adjusted.

According to any embodiments described above, the device may further include a feeder solution sensor in communication with the controller, wherein the feeder solution sensor is arranged to measure electrical conductivity of the feeder solution in the supply tank, and wherein the controller is arranged to adjust voltage applied to the electrodes of the electrolytic cell in response to the measured electrical conductivity so as to maintain a consistent output of the hypochlorous acid solution from the chamber.

According to any embodiments described above, the device may be further arranged so that the supply tank is supported at least partly above the electrolytic cell, and wherein the inlet of the chamber is arranged to receive the feeder solution from the supply tank under force of gravity alone.

According to any embodiments described above, the device may further be arranged so that the electrolytic cell further comprises a gas outlet at a top end of the chamber, in which the gas outlet is arranged to discharge gas from the chamber through the gas outlet and the gas outlet includes a discharge valve arranged to prevent discharge of fluid therethrough in response to a level of the hypochlorous acid solution in the chamber being at or above a prescribed upper limit. The discharge valve may comprise a float movable with an operating level of the hypochlorous acid solution in the chamber in which the discharge valve is arranged to close in response to the float being displaced by the operative level of the hypochlorous acid solution up to said prescribed upper limit.

According to any embodiments described above, the device may be further arranged so that the inlet is located at a top of the chamber, in which the liquid outlet is located at a bottom of the chamber, and in which the electrolytic cell further comprises baffles supported within the chamber between the inlet above and the liquid outlet below. When the electrodes comprise first and second electrode plates mounted parallel to one another within the chamber such that inner side surfaces of the electrode plates define a prescribed electrode gap therebetween and an opposing outer side surface of each electrode plate faces outwardly away from the other electrode plate, the baffles may comprise insulated members mounted adjacent the outer side surface of at least one of the electrode plates.

The baffles may extend upwardly and inwardly from respective opposing side edges of said at least one of the electrode plates towards an upright flow path extending along said at least one of the electrode plates at an intermediate location between the opposing side edges whereby a flow of gasses produced on said at least one of the electrode plates is directly upwardly and inwardly towards the upright flow path by the baffles.

The baffles may protrude perpendicularly outwardly from the outer side surface of said at least one of the electrode plates.

When the electrode plates comprise a perforated sheet material, the baffles may be formed of an insulating material abutted against the outer side surface of said at least one of the electrode plates.

Each baffle may occupy a full depth protruding perpendicularly outward from the outer side surface of said at least one of the electrode plates to a corresponding boundary wall of the chamber.

According to any embodiments described above, the device may be further arranged so that the pump is mounted adjacent to the electrolytic cell, in which the pump and the electrolytic cell are connected by a vibration transmitting structure whereby vibration from the pump assists in releasing gas bubbles from the electrodes to improve electrode to fluid contact efficiency.

According to any embodiments described above, the device further comprise: (i) a conductive member supported in proximity the atomizing nozzle, and (ii) a transformer operatively connected to the conductive member so as to be arranged to charge the conductive member and transfer electric charge to the atomized mist discharged by the atomizing nozzle, wherein the transformer is supported adjacent to the atomizing nozzle and spaced from the controller.

The device may further comprise: (i) an air duct having a duct outlet adjacent to the atomizing nozzle, the air duct receiving the transformer therein; (ii) a fan in communication with the direct so as to be arranged to direct a flow of air through the air duct, about the transformer, and out of the duct outlet in a common flow direction with the atomized mist of the hypochlorous acid solution discharged by the atomizing nozzle whereby the flow of air increases travel distance of the atomized mist and increases electrostatic charge imparted to the mist.

According to any embodiments described above, the device may further comprise (i) a main housing supporting the supply tank, the electrolytic cell and the pump thereon, (ii) a handheld housing supporting the atomizing nozzle and the transformer thereon, and (iii) a flexible line tethering the handheld housing to the main housing, the flexible line being in communication between the pump and the atomizing nozzle to communicate the pressurized flow of the hypochlorous acid solution from the pump to the atomizing nozzle therethrough.

According to any embodiments described above, the device may further comprise a pH sensor operatively connected to the chamber so as to measure a pH value of the hypochlorous acid solution in the chamber, wherein the controller is arranged to cease operation of the pump and actuation of the electrodes of the electrolytic cell in response to the pH value measured by the pH sensor meeting a pH limit stored on the controller.

According to any embodiments described above, the device may further be arranged so that the controller is arranged to cease operation of the pump and actuation of the electrodes of the electrolytic cell in response to voltage and current applied to the electrolytic cell by the controller meeting a prescribed limit stored on the controller.

According to any embodiments described above, the device may further comprise a battery providing electrical power to the electrolytic cell and the pump, wherein the battery, the electrolytic cell, the pump, the controller, and the supply tank are commonly supported on a main housing which is portable and arranged to be carried by a single operator.

According to any embodiments described above, the device may be further arranged so that (i) the electrolytic cell is supported on a main housing, (ii) the supply tank is movable from a mounted position on the main housing to a released position separated from the main housing, and (iii) the supply tank includes a coupling valve supported on an outlet of the supply tank in which the coupling valve is biased to a closed position when the supply tank is in the released position and in which the coupling valve is arranged to be displaced to an open position to allow communication of the feeder solution to the chamber of the electrolytic cell responsive to mounting of the supply tank onto the main housing in the mounted position.

### BRIEF DESCRIPTION OF THE DRAWINGS

The present invention may be better understood, and its numerous features and advantages made apparent to those skilled in the art, by referencing the accompanying drawings. For ease of understanding and simplicity, the common numbering of elements within the illustrations is employed where the same element is in different drawings or figures.

Some embodiments of the present invention are illustrated as an example and are not limited by the figures of the accompanying drawings, in which like references may indicate similar elements and in which:

FIG. 1A depicts a main assembly perspective view of the front of the invention showing the base unit and spray gun according to various embodiments of the present invention.

FIG. 1B depicts a main assembly perspective view of the back of the invention showing the base unit and spray gun according to various embodiments of the present invention.

FIG. 2A illustrates a view of the right side of base unit and spray gun.

FIG. 2B illustrates a view of the back of base unit and spray gun.

FIG. 2C illustrates a view of the left side of base unit.

FIG. 2D illustrates a view of the front of base unit and spray gun.

FIG. 3 depicts an exploded perspective view of the base unit and spray gun system with visibility of the internal components.

FIG. 4 depicts a perspective view of the base unit and spray gun system with the base cover removed for visibility of the internal components.

FIG. 5A depicts view of the front base unit with the cover removed.

FIG. 5B depicts view of the right base unit with the cover removed.

FIG. 5C depicts view of the left base unit with the cover removed.

FIG. 6A depicts front, perspective view of the electrolysis cell and pump assembly in various embodiments described herein

FIG. 6B depicts front, exploded perspective view of the electrolysis cell and pump assembly in various embodiments described herein

FIG. 7A shows a front view of the electrolysis cell showing the electrodes and baffle insulating mounts.

FIG. 7B shows an exploded perspective of the electrolysis cell showing the electrodes and baffle insulating mounts.

FIG. 8A depicts a perspective view of the electrodes and baffle insulator mounts.

FIG. 8B depicts a perspective exploded view of the electrodes and baffle insulator mounts.

FIG. 9A depicts a front view of the automatic gas release valve.

FIG. 9B depicts a perspective exploded view of the automatic gas release valve.

FIG. 10A depicts a perspective view of the spray gun.

FIG. 10B depicts a view of the left-side of the spray gun with the right-side gun housing removed to provide a detail view of the internal component.

FIG. 10C depicts a perspective of the spray gun with the left-side gun housing removed to provide a detail view of the internal components.

FIG. 11 depicts a schematic representation of the hydraulic system for the fluid flows.

FIG. 12A depicts the electronic circuit board.

FIG. 12B depicts the electronic display screen.

FIG. 13 shows an illustrative view of an alternative embodiment in an integrated hypochlorous spraying device that is held in one hand.

### DETAILED DESCRIPTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefits and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

A novel portable, on-demand hypochlorous acid generator and sprayer, contained in a wireless electrostatic backpack system for the purpose of sanitizing and disinfecting are discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention, and is not intended to limit the invention to the specific embodiments illustrated by the figures or description below.

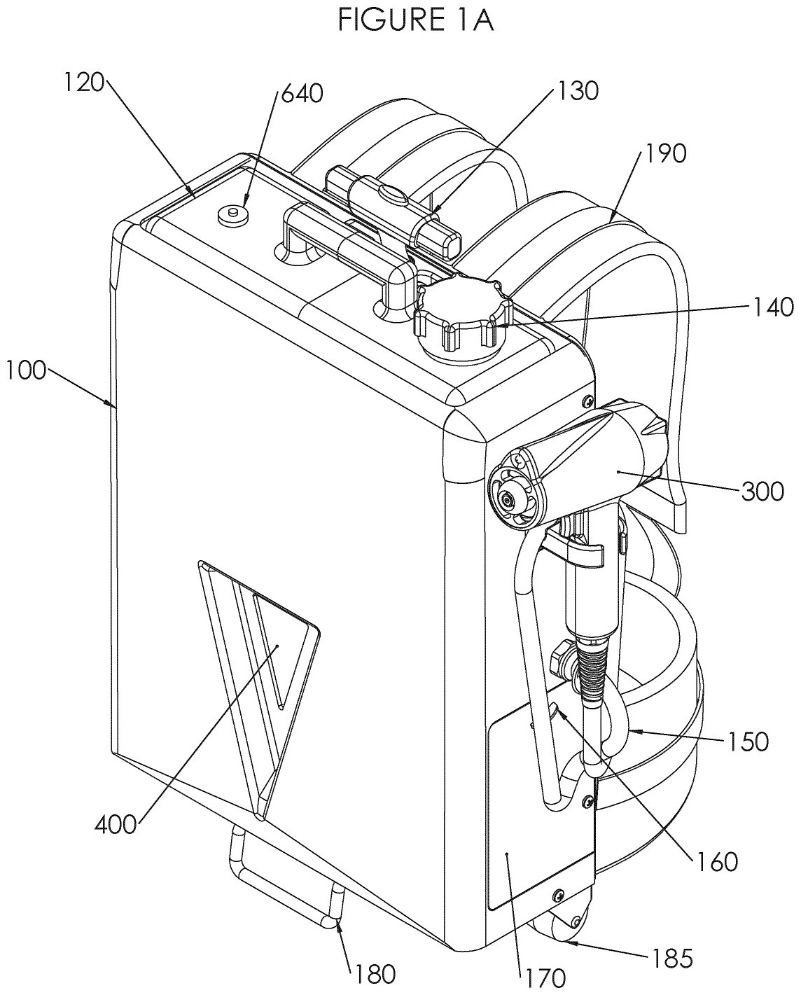

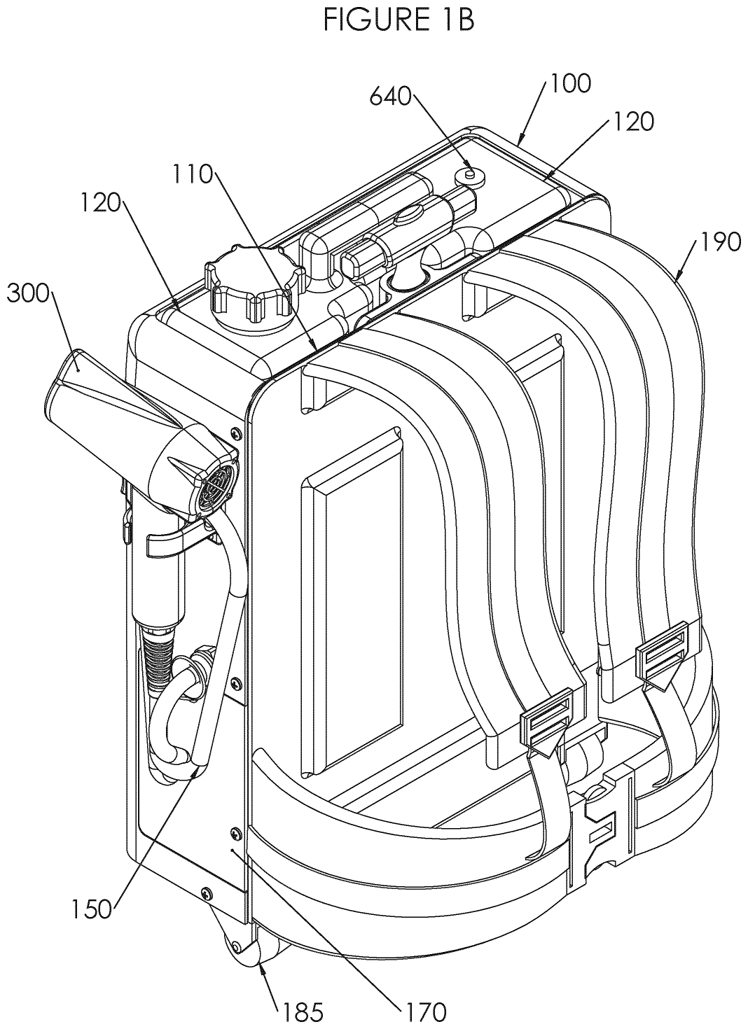

The present invention will now be described by referencing the appended figures representing preferred embodiments. FIGS. 1A & 1B depict a front and back perspective views of an example of a portable hypochlorous generating system in a backpack or handheld, electrostatic sprayer that converts a mixture of water, salt (sodium chloride) and vinegar (acidic acid) to hypochlorous acid for disinfecting and sanitizing surfaces (sometimes referred to as the “apparatus” or the “device”),

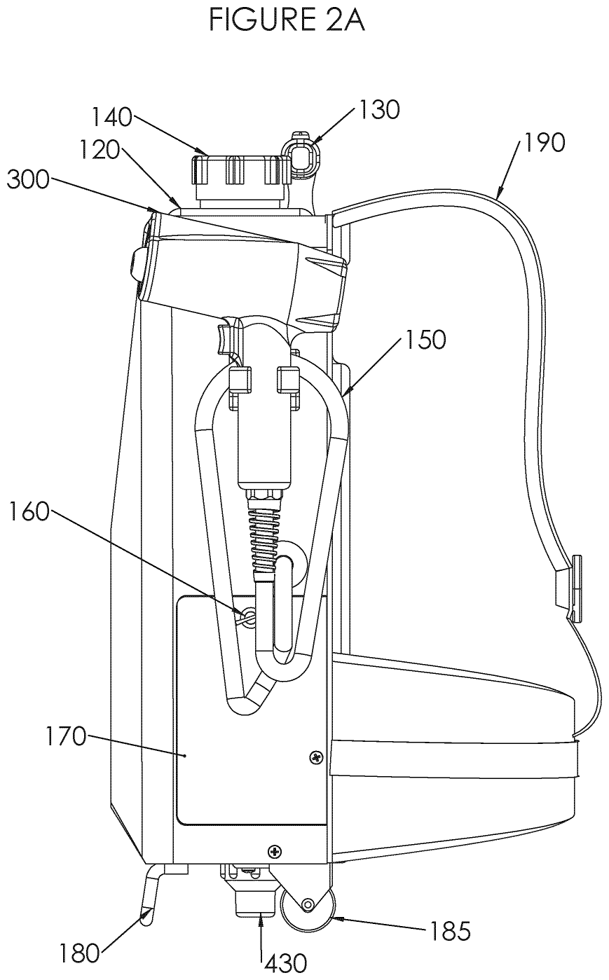

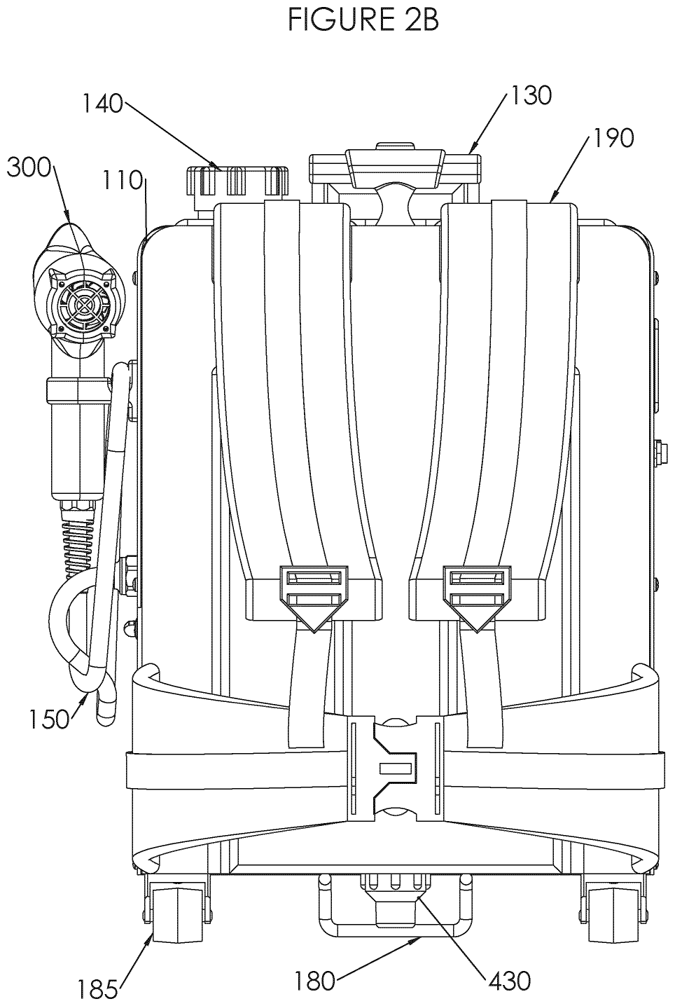

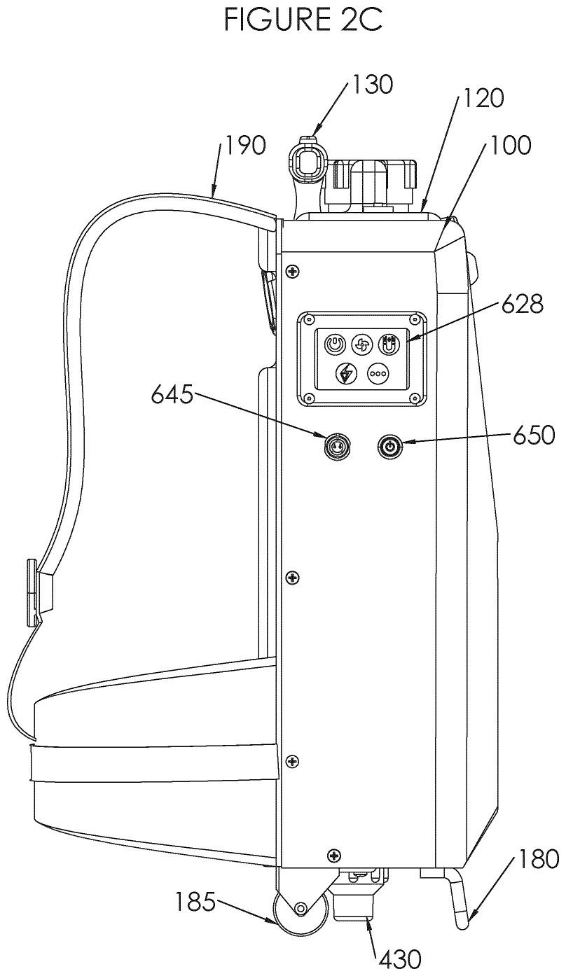

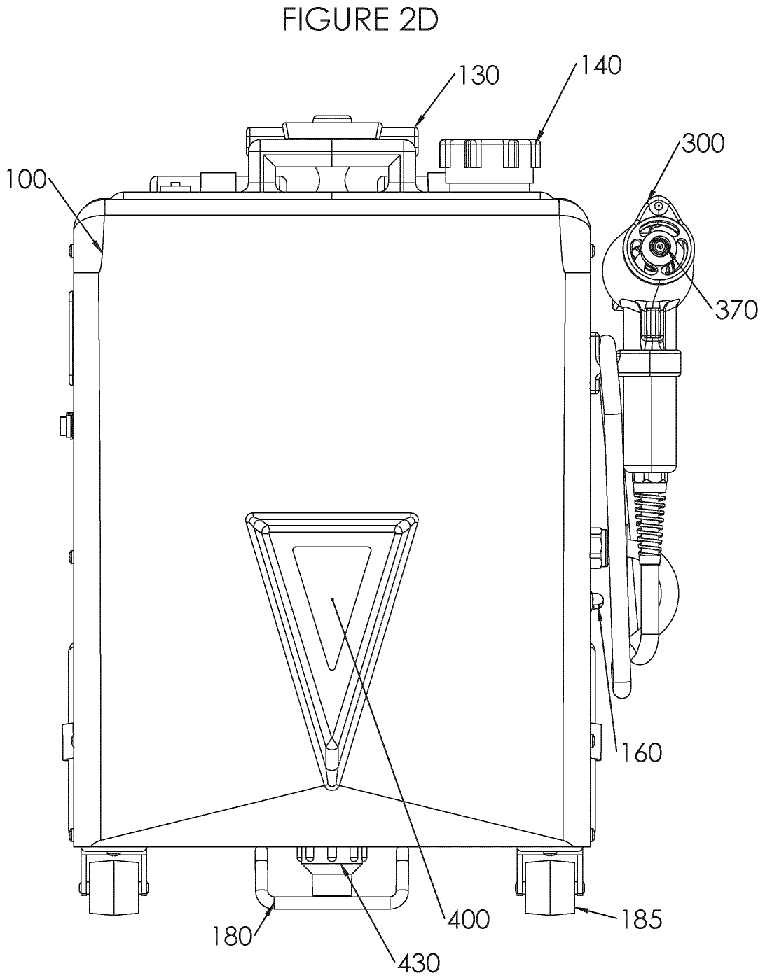

FIG. 1 and FIG. 2 depicts the main assembly views of the portable hypochlorous generating system, electrostatic sprayer. The cover 100 is made from thermoformed ABS or other suitable impact resistant material to protect the internal components. The fluid tank 120 inserts into the top of the device. The tank includes a sealing cap 140 that allows filling and air to flow in or out while preventing fluid from leaking out. The tank 120 is also easily removed for the filling operation and is filled with feeder mixture of approximately 99.7% water, 0.2% salt (Sodium chloride) 0.5% Vinegar (Acidic acid) that is thoroughly mixed. (Other ratios are also suitable.) The backplate 110 made from a corrosion resistant stainless steel, aluminum or other structurally sound plastic, and is used to mount the internal hardware. The backpack harness 190 is made of fabric and padding for the user comfort when the device is worn for spraying.

The telescoping handle 130 allows the device to be transported using the wheels 185 by inclining the device slightly backwards and pulling the handle. A front mounted foot 180 made from aluminum or other structural plastic or material allows the device to remain vertical and stable when unsupported by the user. The device is further connected to the spraying gun 300 that is constructed from ABS. Nylon for impact resistance and durability, with a wire and hose coil 150 linking the electrical wiring and pressure hose to the gun and the device main body.

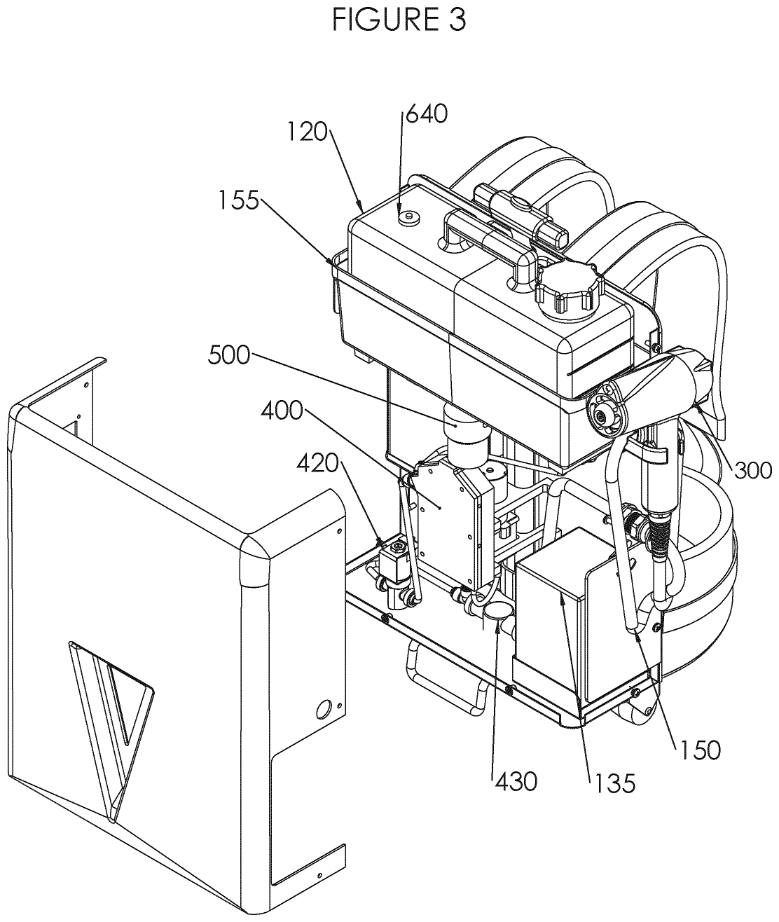

The outer cover 100 includes a visual opening for the electrolysis cell and pump assembly 400 that has LED lighting incorporated for enhanced view of the hypochlorous generating process. A hinged battery access door 170 that includes a door latch 160 provides quick access for the removal and exchange of the 12-volt battery (FIG. 3, 135).

A touch screen displays 628 is mounted to the cover 100 and provides user input for selecting the various hypochlorous concentration settings for food contact 60 ppm, food contact surfaces 100-200 ppm and general surfaces 200+ ppm an also provides communication and error messaging.

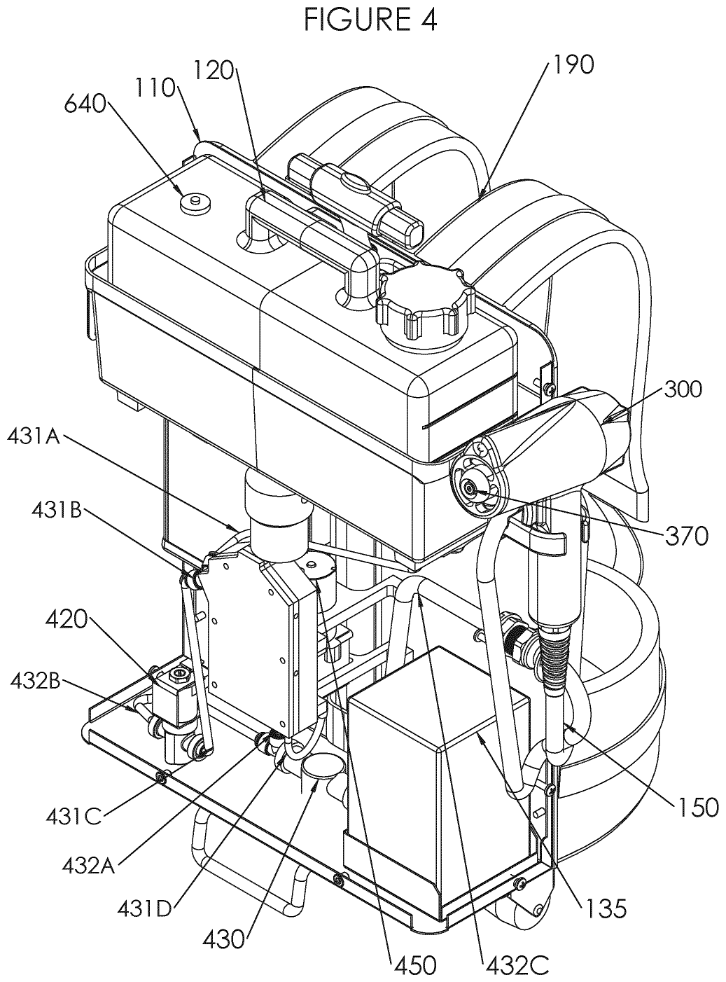

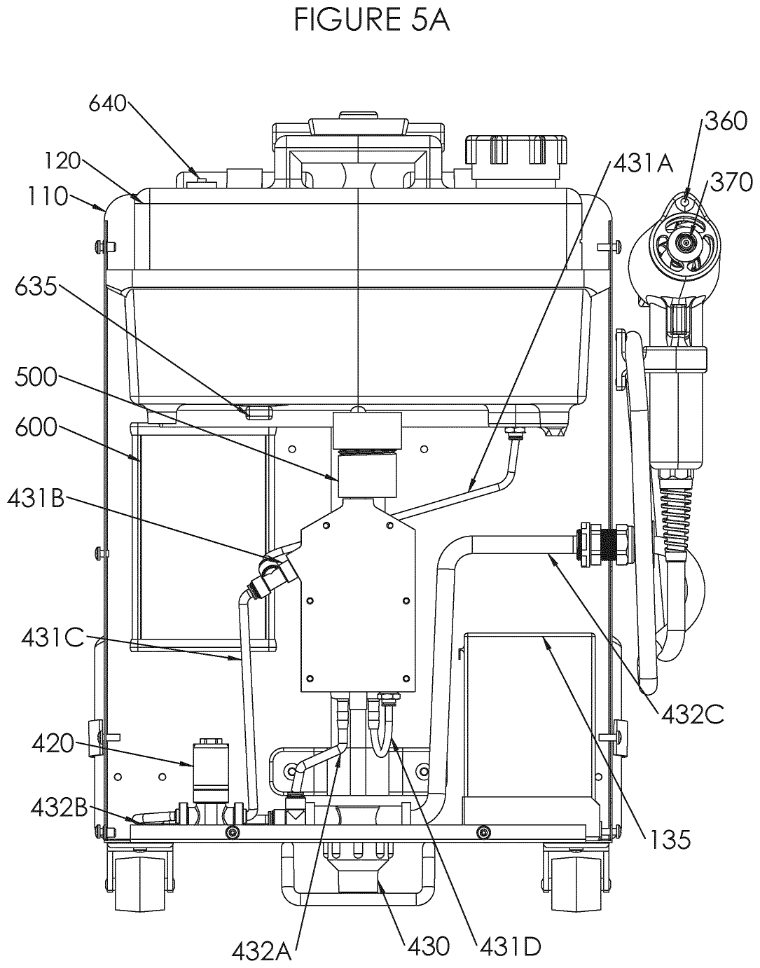

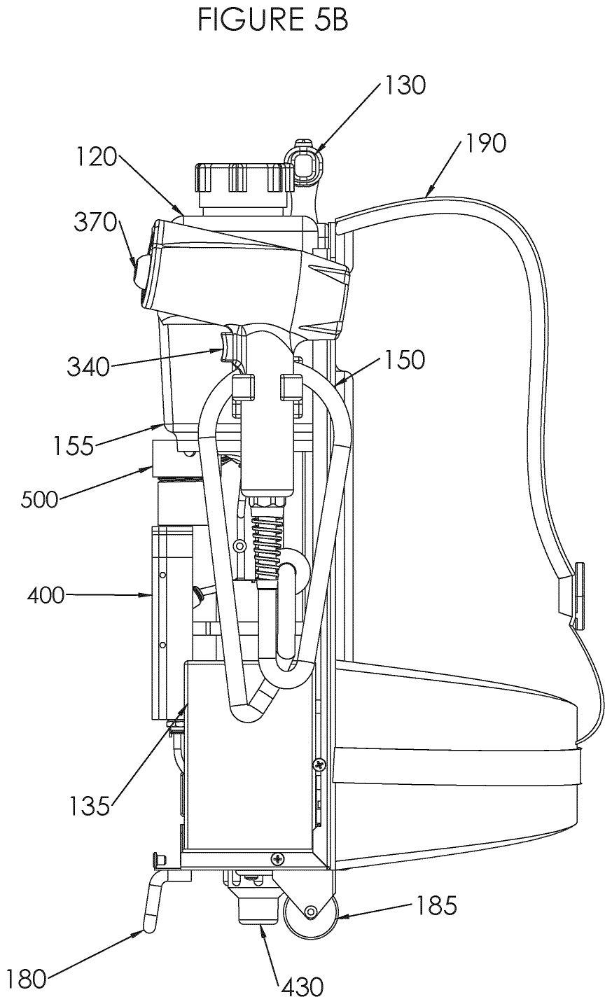

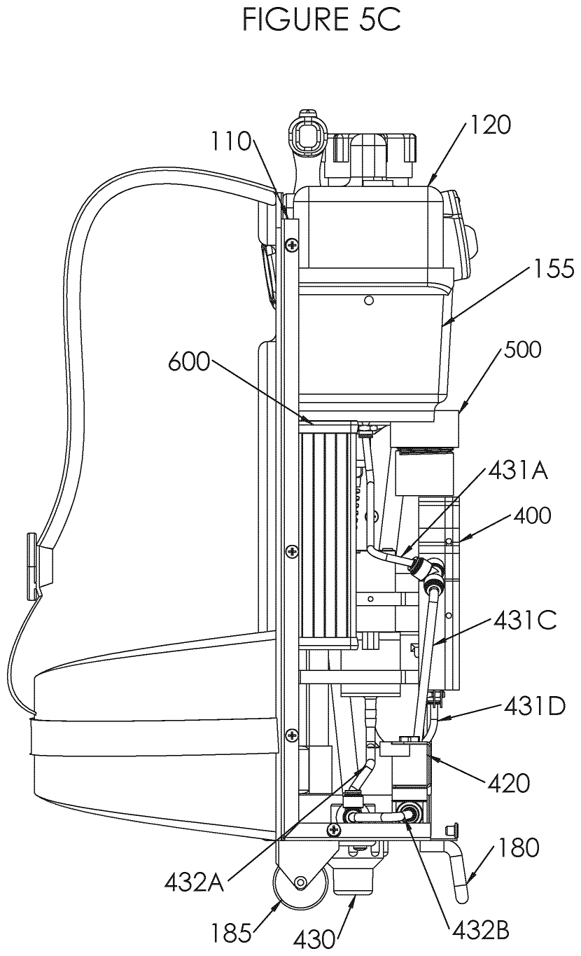

FIG. 3, FIG. 4 and FIG. 5 show the device views with the cover removed. The battery 135 is a 12-volt LIFePO4 lithium iron phosphate with sufficient wattage to support the continuous operation of the device for 3+ hours before needing to recharge. The conversion cell and pump assembly 400 includes an automatic gas release valve 500 that is used to safely release the hydrogen gas created during the electrolysis process without allowing any liquid to pass. The tank 120 docks into the tank holder 155 and connects to the device with a quick coupler.

The solenoid 420 and hydraulic circuit is used to release fluid pressure in the pressurized line. Pressure in the spray line, is released back into the feeder line minimizing the amount of dripping at the nozzle when spraying is cycled on and off. The electrical control box 600 houses and protect the sensitive control board 600B, (used to control monitor the device) from dust, moisture and any accidental contact.

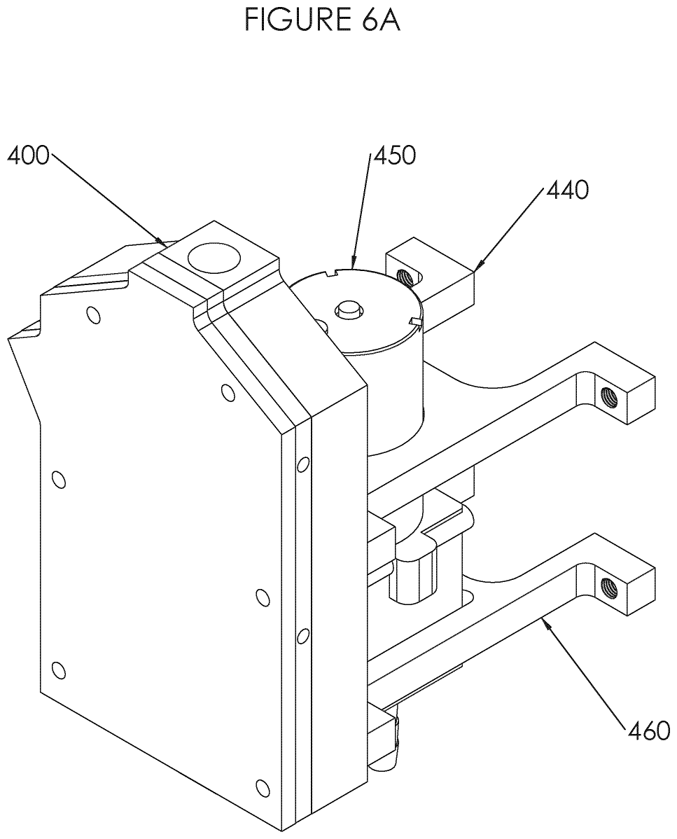

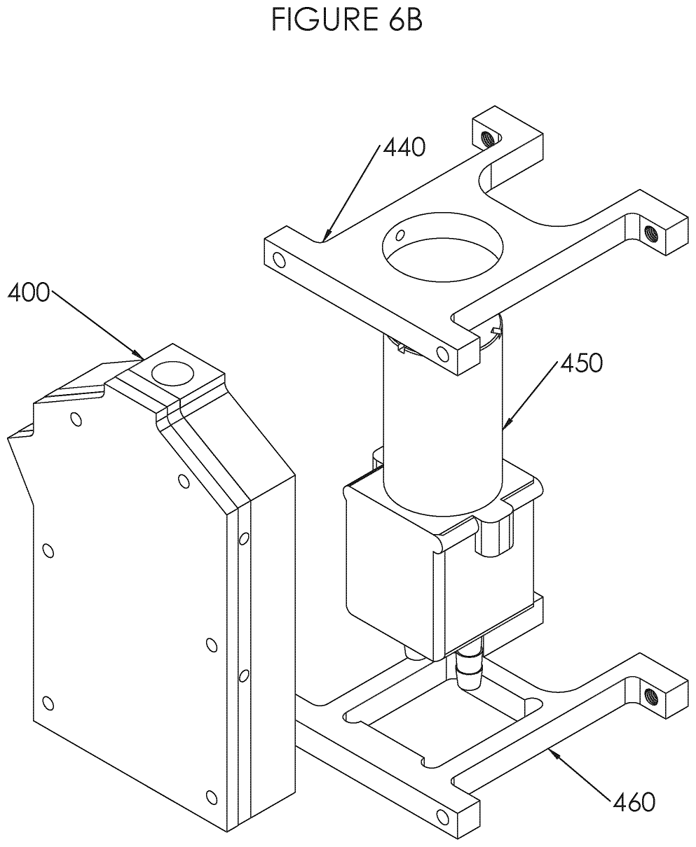

FIGS. 6A & 6B, depict electrolysis cell and pump assembly with the electrolysis cell 400 supplied by the feeder solution that is gravity fed into the chamber. This is constructed of polyvinyl chloride or other suitable corrosion resistance plastic or metallic material and is attached to stand off pump mount 440 and standoff pump mount 460 that contains the fluid pump 450 in the same structure. The pressure pump 450 is used to pressurize the hypochlorous fluid up to 130 PSI. The electrolysis cell assembly 400 is mounted directly in front of the fluid pump 450 with standoff mounts 460 and 440 that are securely connect to the back plate (FIG. 3) 110. The arrangement efficiently transfers the vibration from the fluid pump 450 creating a resonance frequency in the electrolysis cell assembly 400. The vibration causes the hydrogen gas bubbles, that tend to adhering to the electrodes, to quickly release and flow up to the gas exit port at the top of the electrolysis cell 400, increasing the fluid to surface contact area on the electrodes, increasing the hypochlorous acid conversion efficiency.

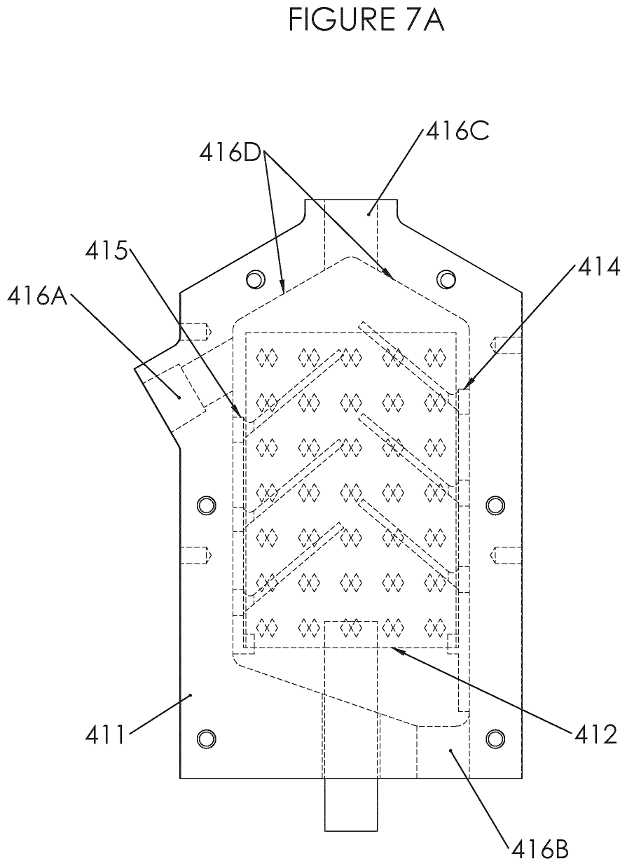

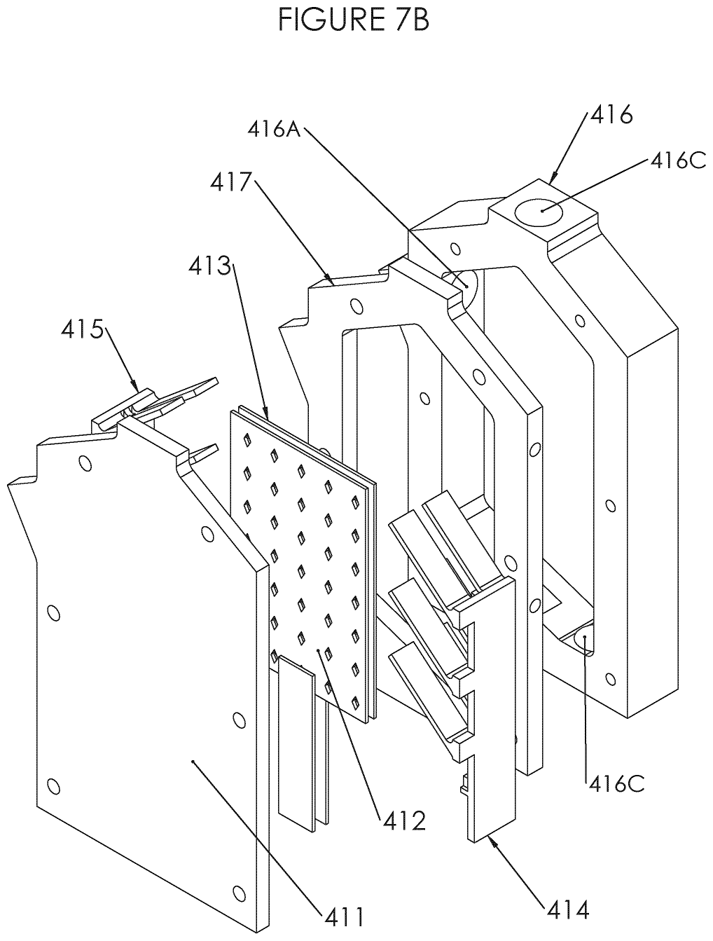

In FIGS. 7A and 7B, the electrolysis cell 400 includes a transparent cover 411 to allow the user to see the conversion process taking place. The cover also contains LED lighting to improve visibility.

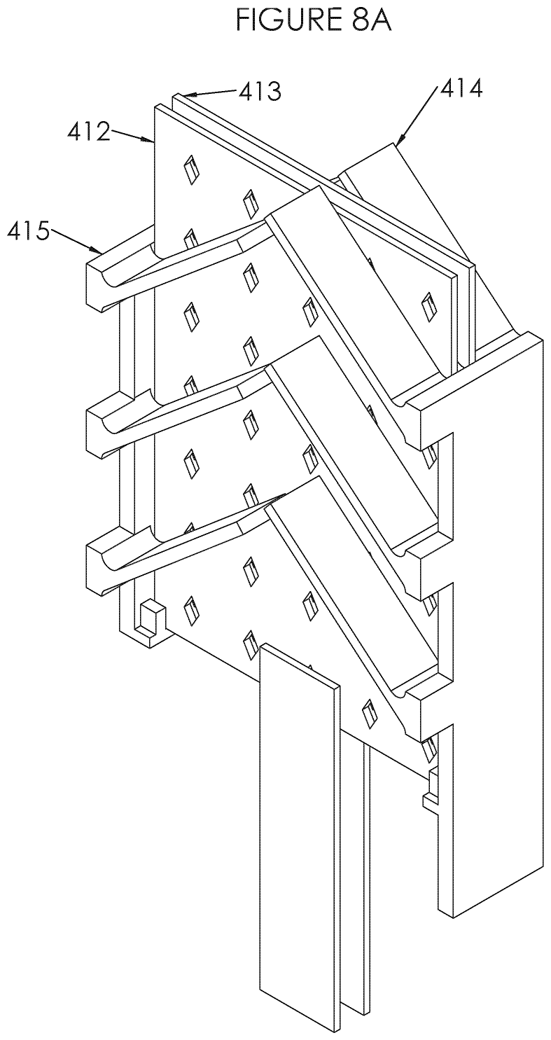

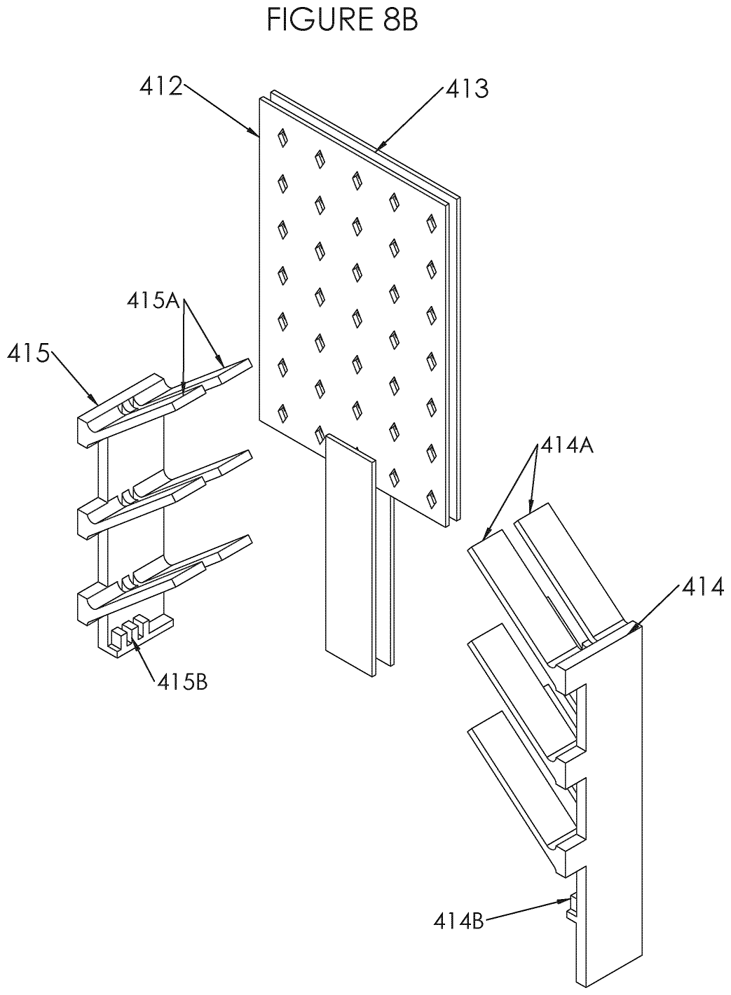

The cell that contains a cathode electrode 412 and anode electrode 413. The anode electrode 412 is constructed from a rectangular perforated mesh but can also be a solid sheet and other profiles, is made from titanium coated with platinum coating or other suitable corrosion resistant conductive material and a cathode electrode 413 that is also constructed from a rectangular perforated mesh but can also be a solid sheet and other profiles and is made from titanium coated with a mixed metal oxide or other suitable corrosion resistant conductive material. The distance between the electrodes is accurately controlled using the baffle insulator, left hand mount 415 and baffle insulator, right hand mount 414 constructed of polyvinyl chloride, ABS, Polyester or other suitable corrosion resistant electrically insulating material, to maintain the desired electrode gap for the electrolysis process. The gap between the electrodes is controlled to 0.04″ with the spacer groves 415B and 414B (FIGS. 8A & 8B) to for efficient conversion and power consumption.

The baffle insulator mounts 414 and 415 also incorporate multiple baffle insulation arms 414A and 415A that are designed to optimize mixing and flow in both the liquid flowing from the upper left inlet port 416A and for the small hydrogen gas bubbles produced during conversion, that flow upwards to the centered gas output port 416C at the top of the electrolysis chamber. Additionally, as the feeder fluid flows down to the bottom of the electrolysis chamber to the hypochlorous outlet port 416B the arms direct the fluid into the center of the electrodes ensuring full fluid conversion. As hydrogen gas is produced, it flows upwards and is directed into the center of the electrode chamber by the baffle insulating arms 414A and 415A. The turbulence created, aids mixing and consistency, as the gas exits the chamber at the upper end of the tapered exit port 416D. The tapper on the port directs the gas flow into the top of the electrode chamber at the gas output port 416C.

The simultaneous liquid and gas flow controlled by the baffle insulator mounts 414 and 415 and associated arms 414A and 415A create mixing swirls that force the feeder solution through the center of the anode electrode 412 and cathode electrode 413 numerous times as it proceeds down to the hypochlorous acid exit port 416B optimizing efficiency and mixing while providing an exit path for the gas. The electrolysis process occurs, on demand and when the sprayer is activated it continuously flows through the electrolysis chamber and is refreshed with feeder solution being processed into hypochlorous acid.

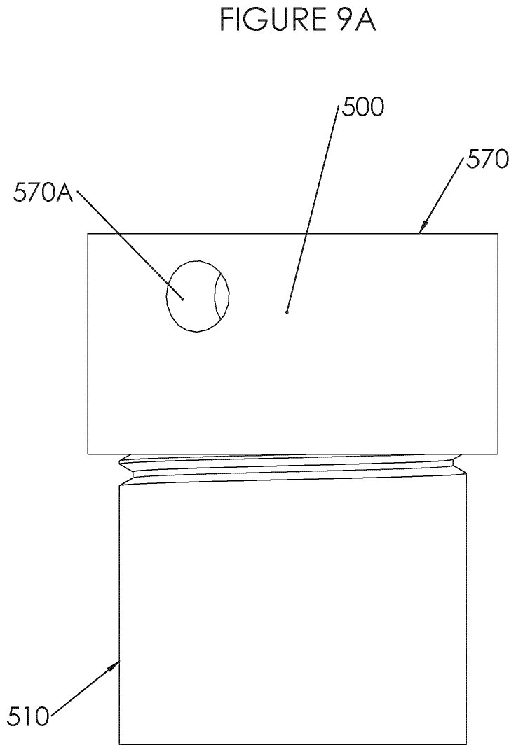

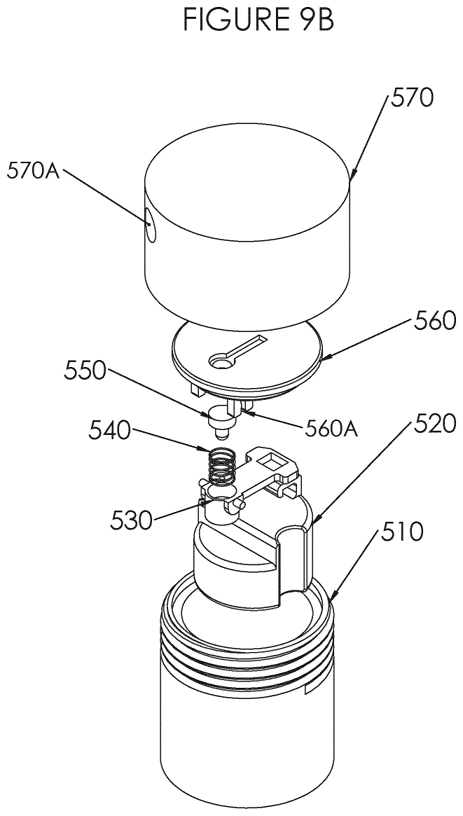

FIGS. 9A and 9B depict the automatic gas relief valve 500, made from PVC or other suitable chemically resistant plastic, consists of a float bowl 510 that fills with gas and liquid, a float 520 that moves up and down according to the fluid level in the float bowl 510. A pivot arm 530 attaches to the inner cap 560 and pivots as the float 520 moves up or down. A sealing element 550 made from a rubber or other suitable sealing material is inserted into the spring 540 and the mechanism comes in contact with the inner cap seat 560.

When the fluid level is high, the sealing element 550 and inner cap 560 seat seal off the release of liquid. When sufficient gases are present in the chamber, the float lowers and releases the gasses without releasing any fluids. The gas then passes up through the outer cap 570 through the release hole 570A mixing safely into the ambient air. A hose attached to the outer cap 570 is another preferred embodiment that directs the gas out to the top or side of the device to quickly dissipate in the air. Another preferred embodiment includes a small fan used to further expel the gas and mix it with the surrounding air.

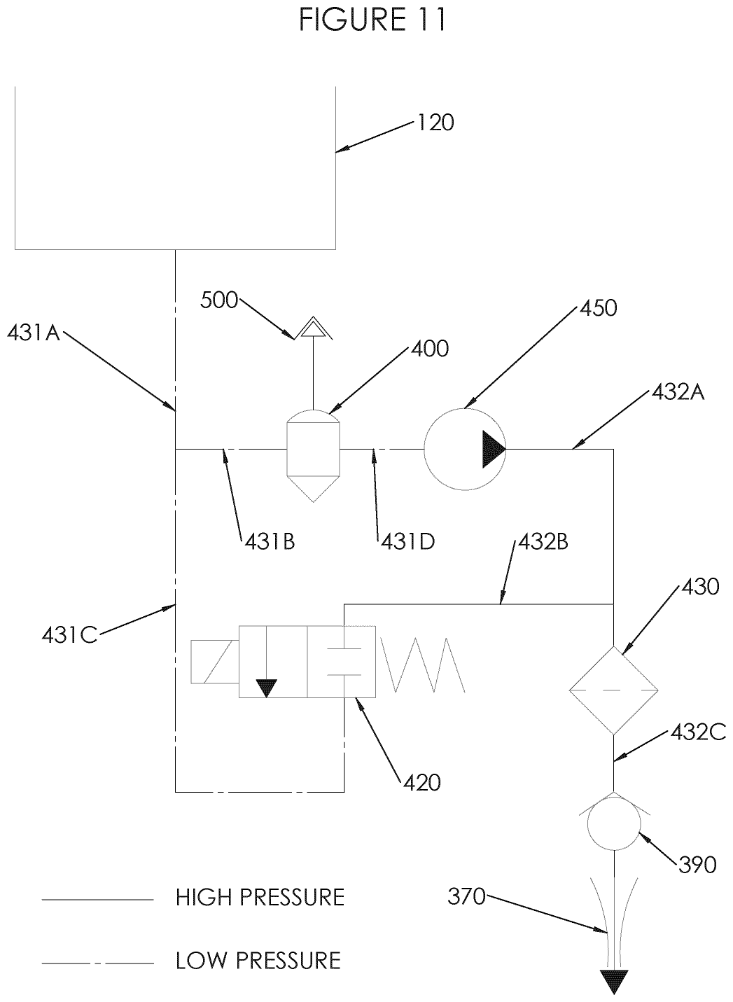

FIG. 11 shows the piping and instrumentation diagram for the fluid flow and control. The feeder fluid gravity flows into the electrode and pump assembly and fills the cavity. During the electrolysis process a small amount of hydrogen gas is produced and is expelled through the automatic gas relief valve. Hypochlorous fluid flows down to the exit port and into the fluid pump, where the pump pressurizes the liquid up to 130 PSI. The fluid then passes through particulate filter and proceeds through a check valve that has a cracking pressure of 1 to 2 PSI. The pressurized hypochlorous acid mixture then exits the atomizing nozzle in an ultra-fine spray. When the spray gun trigger is released, cutting power to the fluid pump, the normally closed solenoid valve is activated, depressurizing the system. The pressure releases back into the gravity fed electrolysis chamber that is under 1 psi. Once the residual pressure is released the cracking pressure prevents the check valve from activating eliminating and dripping at the nozzle.

In FIG. 11, the flow path of the feeder solution starts at the tank 120 that is vented to atmospheric pressure. The electrolysis cell 400 is fed the solution by the low-pressure lines 431A and 431B. The electrolysis cell converts the solution into hypochlorous acid, the hypochlorous acid is then passed through to the pump 450 by the low-pressure line 431D. The pump pressurizes the hypochlorous acid which flows to the filter 430 using the high-pressure line 432A. The hypochlorous acid then flows out of the filter through the high-pressure line 432C to the check valve 390 then to the nozzle 370. The directional control valve 420 acts as a pressure relief by allowing the pressurized fluid 432B to be connected to the low-pressure system by relief line 431C. The directional control valve is activated by the control board 600B.

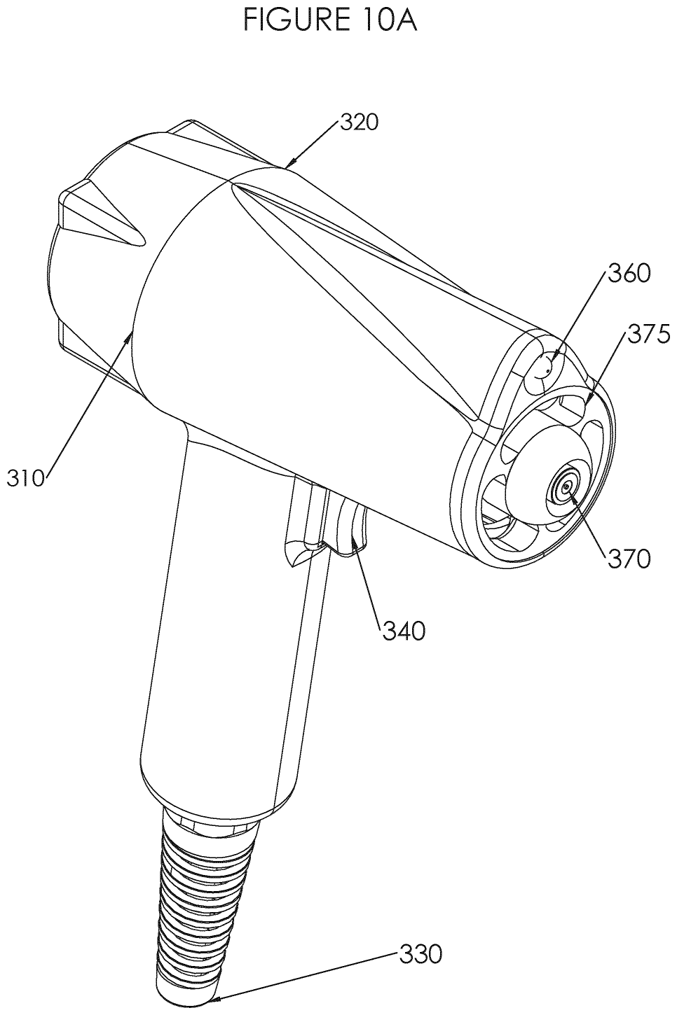

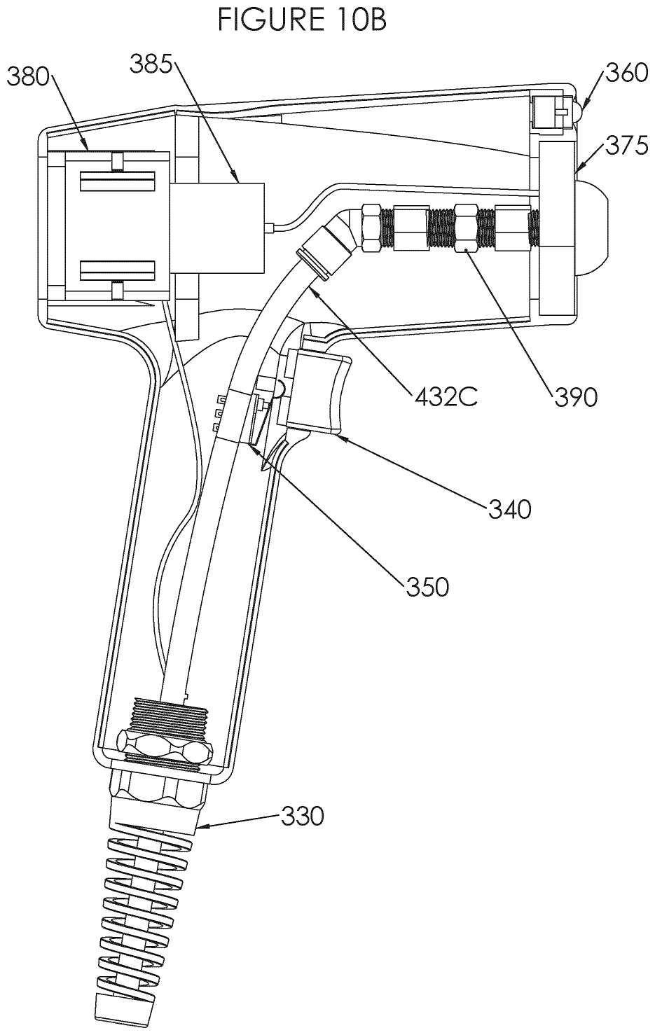

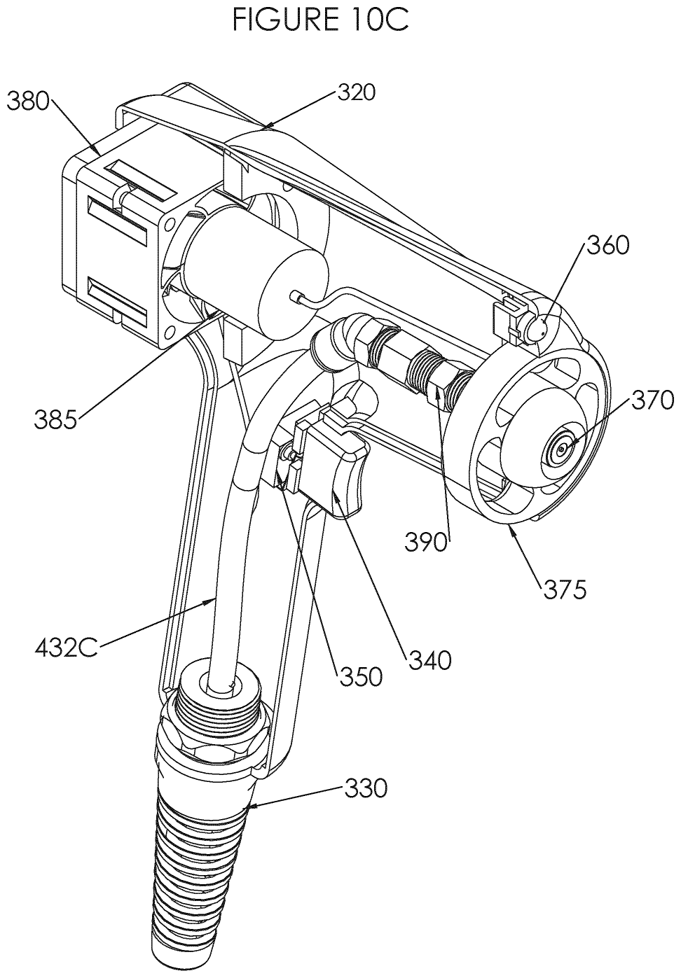

FIGS. 10A, 10B, and 10C. show the spray gun 300 that consists of a left-hand housing 310 and a right-hand housing 320 constructed of impact resistant ABS or Nylon, a strain relief 330 that contains the fluid tubing and wiring for the gun. An activation trigger 340 that connects to the activation switch 350 used to initiate the conversion and spraying. When the switch is engaging the fluid pump 450 is activated, the pressurized fluid is then atomized as it exits the nozzle 370 creating an ultra-fine mist in the range of 40-80 microns. The fan 380 which is activated during the spraying, creates an airflow that mixes with the mist to increase the distance the spray droplets travel, improving coverage of the spray.

The spray droplets pass through an electrically conductive annular ring 375 made of copper, brass or similar conductive material that is electrostatically charged with A 2-20 Kv transformer 385. The transformer is mounted directly in front of the fan 380 and next to the annular ring 375 minimizing the distance of the high voltage line, eliminating interference with other electrical signals and increasing charge efficiency. As the spray droplets pass through the annular ring 375 they pick up a positive charge, but can also alternate between positive and negative charges. Additionally, the air passing over the high voltage transformer and the high voltage line picks up electrostatic charge and mixes with the atomized spray droplets, increasing the overall charge of the mist.

The charged mist droplets, when airborne, are attracted to any neutral or negatively charged surface optimizing transfer and wrap-around effect on the targeted surfaces. An LED light 360 is mounted at the front of the gun to provide task lighting for the user.

As described above a portable hypochlorous acid generating device is provided for generating a hypochlorous acid solution on-demand and spraying the solution as a fine mist for disinfecting purposes.

According to the preferred embodiment, the device generally includes a main housing in the form of a backplate frame 110 supporting a cover 100 releasably thereon such that the hollow interior of the main housing can be accessed when the cover is removed. Within the hollow interior of the main housing there is supported the electrolytic cell 400 which is supplied with the feeder solution from the supply tank 120 supported above the cell to enable feeding of feeder solution from the tank 120 to the interior chamber of the cell 400 under force of gravity alone.

A pump 450 is also supported within the hollow interior of the main housing and is connected adjacent to the electrolytic cell 400 by a vibration transmitting structure 440 and 460 such that vibration from the pump is transmitted through the structure to the electrodes of the electrolytic cell 400 to assist in releasing gas bubbles from the electrodes which form during electrolysis for improving electrode to fluid contact efficiency. The pump 450 is in communication with the liquid outlet 416B of the electrolytic cell 400 to produce a pressurized flow of the hypochlorous acid solution drawn from the electrolytic cell.

A controller 600B is also supported within the hollow interior of the main housing in the form of a printed circuit board having a computer memory storing programming instructions thereon and a processor for executing the programming instructions to perform the various functions described herein. A battery 135 is also supported within the hollow interior of the main housing to provide electrical power to the controller 600B, the pump 450 and the electrolytic cell 400, as well as various additional sensors and electrical components described further herein.

The entirety of the main housing and the various components supported thereon including the battery, the electrolytic cell, the pump, the controller, and the supply tank is sized and arranged to be carried by a single operator so as to be readily portable.

The hypochlorous acid generating device according to the preferred embodiment further includes a handheld housing in the form of the spray gun 300 which is separate from the main housing while being connected by a flexible line 150 functioning as a tether in connection between the main housing and the handheld housing. The handheld housing 310 and 320 supports the atomizing nozzle 370 thereon in communication with the pump 450 through the flexible line 150 such that the atomizing nozzle is arranged to receive the pressurized flow of the hypochlorous acid solution from the pump and discharge the solution as an atomized mist from the atomizing nozzle in an axial direction of the nozzle.

The handheld housing 310 and 320 further supports a trigger switch 340 and 350 which is suitably positioned for alignment with an index finger of the user when a handle portion of the handheld housing is gripped in a first of a user. Actuation of the trigger switch by the user generates an activation signal communicated to the controller board 600B which serves to simultaneously (i) actuate the pump to pump the hypochlorous acid solution from the electrolytic cell 400 to the atomizing nozzle 370 and (ii) actuate the electrodes 412 and 413 of the electrolytic cell 400 to generate hypochlorous acid. Drawing the hypochlorous acid solution from the electrolytic cell using the pump is sufficient to cause new feeder solution to be fed by gravity from the supply tank into the chamber of the electrolytic cell to replace the solution that is drawn by the pump in a continuous flow operation during actuation of the trigger switch.

The handheld housing 300 further includes an air duct communicating through the housing in the axial direction of the atomizing nozzle from an air inlet supporting a fan 380 therein to an air outlet surrounding the atomizing nozzle 370. The fan is arranged to direct a flow of air through the air duct in a flow direction which is coaxial with the longitudinal axis of the atomizing nozzle such that the air flow exiting the air duct and the discharge of atomized mist of hypochlorous acid solution discharged by the atomizing nozzle are directed out of the handled housing 300 in a common direction whereby the flow of air increases travel distance of the atomized mist while also increasing electrostatic charge imparted to the mist as described in the following.

To assist in imparting an electrostatic charge to the atomized mist, a conductive ring 375 is supported coaxially about the atomizing nozzle in which the conductive ring is charged by a transformer 385. The transformer 385 is positioned within the air duct in the handheld housing 300 at an intermediate location between the fan 380 and the conductive ring at the atomizing nozzle. In this manner, the airflow through the housing by the fan 380 is directed through the duct surrounding the transformer 385 prior to exiting through the air outlet that surrounds both the atomizing nozzle and the conductive ring 375 so that additional static charge can be imparted to the mist by the airflow surrounding the transformer. In this manner the transformer 385 is positioned within the handheld housing 300 in proximity to the atomizing nozzle and other components of the handheld housing, while being located remotely from the majority of the other electrical components and the controller 600B supported within the main housing so as to minimize electrical interference between the transformer and other electrical components of the device.

The device further includes a user input device in the form of a touchscreen display 628 which allows various user selections to be input into the controller. In one instance, the user input device 628 is arranged to receive a selected acid concentration input from the user 628D. The controller is arranged to adjust the voltage applied to the electrodes of the electrolytic cell 400 to correspond with the selected acid concentration input by the user into the controller board 600B. The concentration of hypochlorous acid within the hypochlorous acid solution is then adjusted by the adjustment to the applied voltage so that the concentration corresponds with the selected acid concentration input by the user as the device continues to operate. The display 628 further allows the user to activate the fan 628B, electrostatics 628C, and other device details 628E.

As further described herein, a feeder solution sensor 635 can be mounted at various locations in communication with the feeder solution either in the supply tank, or in the feed line from the supply tank to the inlet 416A of the electrolytic cell 400 for measuring the electrical conductivity of the feeder solution in the supply tank. As the electrical conductivity of the feeder solution will vary depending upon concentration of electrolyte within the feeder solution, the controller can be arranged to adjust the applied voltage to the electrodes in response to the measured electrical conductivity to maintain a consistent output of hypochlorous acid being generated within the chamber despite varying concentrations of electrolyte which might be present in the feeder solution. Measurement of electrical conductivity can also be accomplished by using the electrolysis electrodes measuring the resistance of the liquid between the electrodes.

A pH sensor 640 can also be operatively connected to the tank for communication with the solution in the tank to measure a pH value of the feeder solution. In this instance, the controller can be arranged to cease the operation of the pump 450 and the actuation of electrical power to the electrodes 412 and 413 of the electrolytic cell 400 in response to the pH value measured by the pH sensor meeting a pH limit stored on the controller. This can include a pH value that exceeds an upper pH limit or a pH value that falls below a lower pH limit.

The controller board 600B is also further arranged to monitor voltage and current applied to the electrolytic cell by the controller, and then compare the monitored voltage and current in real time to prescribed upper or lower limits stored on the controller for each of the voltage and the current. If either one or both of the monitored voltage or the monitored current exceeds the respective upper limit or falls below the respective lower limit, the controller can respond by ceasing operation of the pump and the actuation of electrical power to the electrodes of the electrolytic cell to prevent a dangerous condition occurring.

Turning now more particularly to the construction of the electrolytic cell 400 in further detail, the cell 400 comprises a chamber fully surrounded by boundary walls 416, 417, and 411 to enclose the chamber such that the chamber is suitable for containing the hypochlorous acid solution therein. An inlet 416A communicates through the boundary walls 416 adjacent a top end of the chamber and receives the feeder solution from the supply tank 120. The liquid outlet 416B communicates through the boundary walls 416 at the bottom end of the chamber for communication with a fluid line 431D in communication between the electrolytic cell and the inlet of the pump 450. A gas outlet 416C is located at the top of the electrolytic chamber at a location spaced above the inlet 416A.

A gas discharge valve 500 is mounted within the gas outlet 416C to control the discharge of fluid from the electrolytic chamber through the gas outlet. The gas discharge valve is configured to allow discharge of gas from the chamber through the gas outlet while preventing discharge of gas or liquid therethrough in response to a liquid level of the hypochlorous acid solution in the chamber being at or above a prescribed upper limit of the gas discharge valve. More particularly, the gas discharge valve includes a float operatively connected to the valve in which the float is movable with an operating level of the hypochlorous acid solution within the chamber and is further arranged to close the discharge valve in response to the operating level of liquid reaching the prescribed upper limit. In this instance, when gas builds up at the top of the chamber so that the liquid level falls below the upper limit, the gas discharge valve opens to release gas from the chamber, which in turn allows free flow of feeder solution from the supply tank 120 to enter the chamber under force of gravity. As the entry of feeder solution into the electrolytic chamber raises the liquid level up to the prescribed upper limit of the gas discharge valve, the gas discharge valve will close, thus preventing any escape of liquid or gas through the gas discharge valve until the liquid level falls below the prescribed upper limit once more.

The electrolytic cell 400 further includes two electrode plates 412 and 413 of opposed polarity to define one anode and one cathode, in which the plates are supported by insulating material within the chamber to lie parallel to one another with inner side surfaces of the plates face inwardly one another at a prescribed electrode gap spacing therebetween while opposing outer side surfaces of the plates each face outwardly and away from the other electrode plate. A set of first baffles 414 and a set of second baffles 415 formed of insulating material are mounted in abutment against the outer side surfaces of the electrode plates to direct the flow of gas bubbles towards the central gas outlet 416C while also requiring the liquid in the electrolytic chamber to follow a sinuous path from the inlet 416A at the top to the liquid outlet 416B at the bottom to encourage mixing of the solution and encourage a more even concentration of hypochlorous acid throughout the chamber in the electrolytic cell 400.

The electrode plates 412 and 413 span vertically a majority of the height of the electrolytic chamber and have a width in a lateral direction between opposing side edges of the plates that spans substantially the full width of the electrolytic chamber in the lateral direction. The baffles 414 and 415 comprise flat plates or arms which protrude perpendicularly outward from the outer side surface of the electrode plates 412 and 413 respectively to span a full depth in a direction perpendicularly to the plates from the corresponding outer side surface of the plate to a corresponding one of the boundary walls 416 of the electrolytic cell.

At the outer side surface of each electrode plate, the baffles 414 and 415 are each supported to extend laterally inwardly from one of the side edges of the electrode plate at an inward and upward slope to an inner end of the baffle terminating at a central location in the lateral direction of the electrode plates. Baffles extending from a first side edge of the plate alternate in elevation with baffles extending from the opposing second side edge, with all of the plates terminating along an upright central flow path extending upwardly along the outer side surface of the electrode plate at a laterally central location. In this manner, any bubbles forming on the outer side surfaces of the electrode plates will tend to rise up along the outer side surfaces of the baffles to be directed upwardly and inwardly by the slope of the plates to the central flow path where the bubbles subsequently flow upward towards the gas outlet 416C. The flow of bubbles in turn draws a flow of surrounding solution with the bubbles towards the central flowpath and the gas outlet. The alternating elevation of (i) baffles originating from the first side edge of the electrode plate and (ii) baffles originating from the second side edge of the electrode plate results in the upright flowpath following a sinuous path around the inner free edges of the baffles from the bottom to the top of the electrolytic cell.

In opposition to the upward flow of bubbles, new feeder solution entering the cell inlet 416A at the top of the chamber and navigating towards the liquid outlet 416B at the bottom of the chamber as it forms a hypochlorous acid solution must also navigate around the baffles to encourage mixing of the solution. The combination of the baffles which span the full depth from the electrode plates to the corresponding outer boundary walls of the chamber together with the electrode plates being formed of a perforated material encourage the flow of liquid solution to pass in and out of the perforations in the plates between adjacent baffles to replace liquid being drawn upwardly with the flow of bubbles. The overall result of the liquid solution flowing upward by the bubbles following the slope of the baffles together with replacement liquid solution generally flowing downward results in many turbulent flows within the chamber to encourage a high degree of mixing of the solution and homogeneity of the hypochlorous acid concentration within the solution prior to the solution being discharged through the outlet 416B at the bottom of the chamber.

Use of the device begins by providing the prescribed feeder solution within the supply tank which then automatically fills the electrolytic cell with feeder solution up to the prescribed upper limit of the gas discharge valve. Upon initial actuation of the trigger switch 340 and 350, the controller actuates the pump 450 and the electrolytic cell 400 simultaneously at a voltage level corresponding to the concentration level set by the user and adjusted according to the measured electrolyte concentration in the feeder solution. The rate of production of hypochlorous acid is proportional to the rate of removal of solution from the chamber by the pump so that over an elapsed operating time for example in the order of 1 to 2 minutes, the concentration of hypochlorous acid within the solution emitted from the atomizing nozzle will correspond to the concentration level set by the user through the input device 628. If the concentration level is adjusted by the user, upon subsequent triggering by the user, the controller will apply the new prescribed voltage to the electrodes of the electrolytic cell to produce a new concentration of acid within the solution in the cell once the prescribed operating time has again elapsed for example in the order of 1 to 2 minutes.

The device continues to operate the pump and the electrolytic cell simultaneously while the trigger is continued to be activated by the user. Upon release of the trigger, operation of the pump and the electrolytic cell cease together. To prevent continued discharge of the pressurized flow through the atomizing nozzle, a relief line 431C is provided in communication between the outlet pressure line 432A that supplies the pressurized flow from the pump to the atomizing nozzle 370 in the supply line 431B that delivers solution from the electrolytic cell inlet 416A. This connection to the valve to the from the main outlet pressure line 432A is done by A control valve 420 operated by the controller board 600B is connected in line with the relief line to allow flow through the relief line when opened, but blocks flow through the relief line when closed. Upon ceasing actuation of the trigger, the controller will open the control valve which in turn allows any pressurized fluid in the outlet line to be returned to the electrolytic cell or the supply line connected to the cell inlet 416C, thereby depressurizing the outlet line.

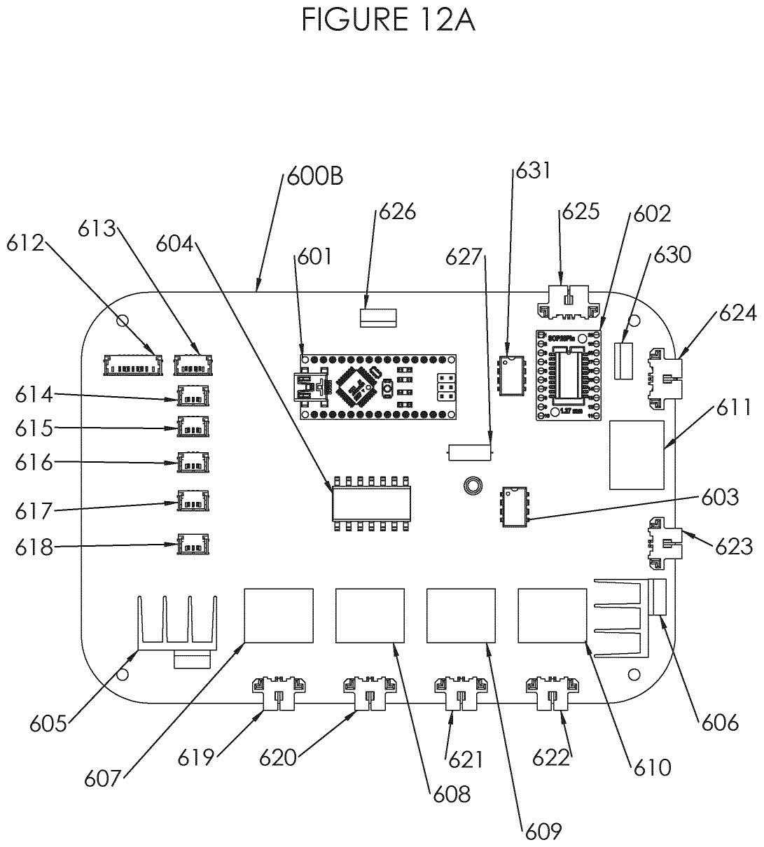

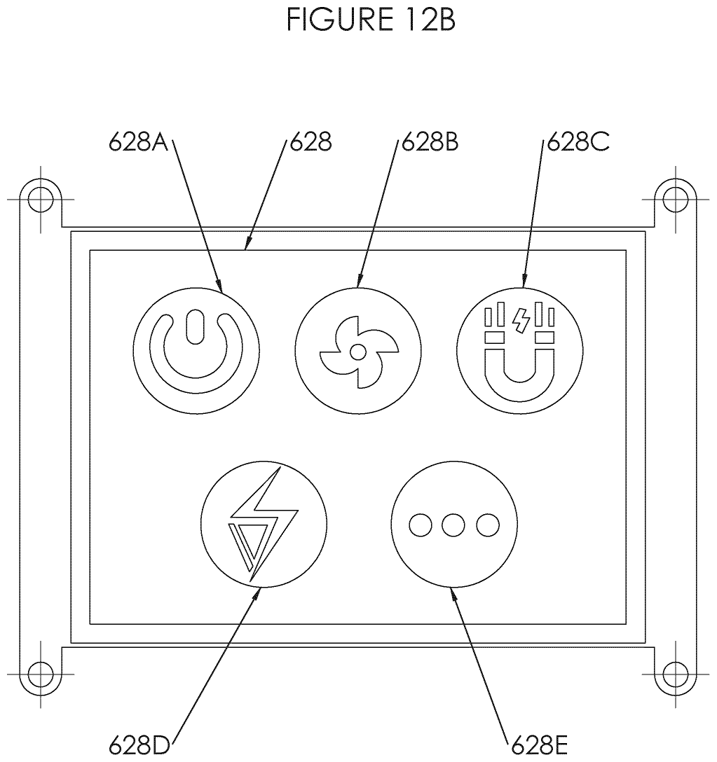

FIG. 12A shows the electrical board 600B and component placement of various components listed in the following.
601: Logic controller
602: Adjustable DC Buck-Boost Converter—Used as main power source to Electrolysis cell
603: 2 Channel Operational Amplifier (Op Amp)
604: 7 Channel Darlington Pair integrated circuit (IC)—Used for control of board relays
605: Fixed DC 5V Convertor—Used for Relay, Arduino and sensor power
606: Fixed DC 3.3 Convertor—Used for Power to High voltage Transformer for electrostatic
607: Output Relay—Fan
608: Output Relay—Pump
609: Output Relay—Water Valve
610: Output Relay—Used for 606 3.3V DC Converter
611: Output Relay—Used for 602 Adjustable DC Buck-Boost Converter
612: Serial Peripheral Interface (SPI) Bus Output Connector-Used for touch screen
613: Inter-Integrated Circuit (I2C) Bus, Power and Ground Connector-Used for Touch Screen input
614: Sensor Input Connector—Current Sensor
615: Sensor Input Connector—Spare
616: Sensor Input Connector—Spare
617: Sensor Input Connector—Gun Trigger Input
618: Sensor Input Connector—Ultra Sonic Level Sensor
619: Output Connector—Used for 607
620: Output Connector—Used for 608
621: Output Connector—Used for 609
622: Input Connector—Used for power and ground board input
623: Input/Output Connector—Used for Power/ground Input and 3.3 Output to Electrostatic Board
624: Input/Output Connector—Used for Power/Ground Input and 602 Output
625: Input Connector—Ground
626: NPN Transistor
627: Low Pass Filter (Resistor and Capacitor—Used for Pulse Width Modulation (PWM) Filtering
630: NPN Transistor
631: Hall Effect Current Sensor

FIG. 12B shows the touch screen and the home page of said touch screen including the following components: 628: Touch Screen; 628A: Device On/Off Icon; 628B: Fan On/Off Icon; 628C: Electrostatics On/Off Icon; 628D: Hypochlorous concentration setting icon; and 628E: Device Details Icon.

The main electrical board 600B is used for electrical output control for the backpack system. 601 is the logic controller that has various programmed functions.

When the system is first turned on the logic controller 601 boots up and initiates several functions, the first being the communication protocols that connect to the touch screen 628 through connectors 612 and 613. After the screen communication has been initiated the controller 601 provides information on the display, the main being input control buttons and status information 628. The information displayed to the screen is done through the communication bus while the touch screen feedback is done through a secondary communication bus. Part of the Startup sequence is checking the system for errors and tank level using the ultrasonic sensor 635 connected through 618. If the tank is low, the logic controller with display the low status to the touch screen.

The system will sit idle until the user selects the parts per million (PPM) concentration (60, 100, 200, 300 PPM) as well as enable the system, which is all done through the touch screen 628. The selection of the PPM dictates the power setpoints that the logic controller will maintain during operation. Once the system and setpoints have been enabled, the system will be activated once user pulls the trigger connected through 617.

With the trigger pulled, logic controller 601 will provide a Pulse Width Modulation (PWM) signal that will first be filtered by the low pass filter 627, this is to convert the PWM to an analog voltage, functionally acting as a digital analog converter (ADC). This voltage output then enters one of channels of the operational amplifier (Op Amp) 603, this channel is used to increase the gain of the voltage output to allow for higher voltage to be outputted. This channel output then goes into the remaining channel of the OP Amp 603, this channel's output provides high imprudence and effective electrical isolation to the NPN transistor 630A connected to the output of the second channel of the Op Amp. The NPN transistor allows adjustment of the voltage trim on the 602 DC buck-boost convertor. Effectively this circuit allows changes in the PWM signal to control the voltage output of 602. The output of 602 then passes through the Hall effect current sensor 631. Finally, this output passes through the activated relay 611 and connector 624 to the electrolysis cell.

In order to maintain the power output of the electrolysis cell, the PWM output from the logic controller 601 is calculated based on a Proportional Integral Derivative (PID) loop programmed into the logic controller 601. The setpoint for the PID loop is the predetermined power correlated to the PPM concentration, this relationship was done experimentally through a series of tests and is done as an equation programmed into the logic controller. The main input for the PID loop is the calculated power (wattage) taken from output voltage of 602 and the current sensor reading of 631. As there are various variables that dictate the power draw of the electrolysis cell, this control loop and sensors allow for these variations and can maintain the setpoint power to the cell, effectively controlling the PPM being produced.

As the logic controller is running the PID control loop is also activates the main pump 608, fan 607, electrostatic power 610 and electrolysis power 611 relays. The controller output does not have enough power itself to control the relays and runs the risk of the flyback effect (Back current from relay coil). To eliminate these issues and concerns, the output from the logic controller 601 first passes through the Darlington Pair Integrated Circuit 604. When the output is activated, the transistors within 604 open, allowing the current to flow from the 5V DC convertor 605 through the activated relays to ground; this closes the contacts of the relays and allows for the outputs to be turned on.

When the user releases the trigger switch connected to 617, immediately the logic controller will shut off all outputs relays 607, 608, 610 and 611, this will shut off the pump, fan, electrostatic and electrolysis cell. The controller will also pulse the output to the relay 609 (water valve 420). This depresses the main sprayer line from the pump to the gun nozzle.

The end user can also control certain functions of the system through the touch screen 628, these include the PPM setting, fan output and electrostatic output. If the user does not enable the fan or the electrostatic on the touch screen, neither will activate during operation/spraying.

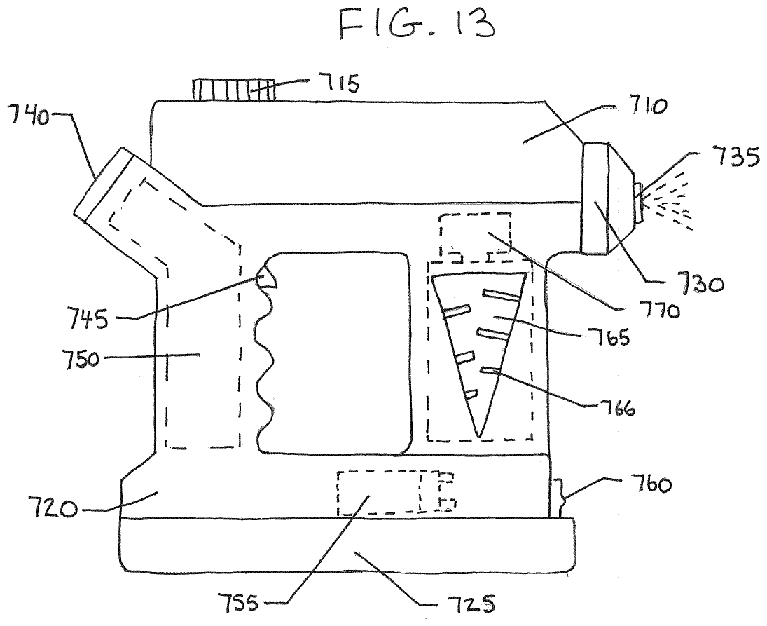

FIG. 13 shows additional preferred embodiments incorporating the novel features of the device that are integrated into a configuration that is held and operated with one hand. The various components are reduced in size and arranged so that the system's portability is further improved for holding and reduced weight for operation in a single hand while spraying.

The feeder fluid of sodium chloride and acetic acid is filled in tank 710 at the top of the handheld hypochlorous acid generator at filling cap 715. The feeder solution flows downward, filling the electrolysis cell 765 previously described. When the device is turned on via touch screen 740 and activated by trigger 745, the voltage is applied via the controller to the electrolysis cell 765 and the pressure pump 755 to initiate the conversion of the feeder mixture and spraying of Hypochlorous acid. Power is supplied from a swappable 12-volt lithium battery 725. During this process, hydrogen gas is produced and flows upward, and exits the cell at the float gas release valve 770 mounted at the top of the cell. The feeder liquid flows from the top down through the electrolysis cell 765 and insulator and mixing baffles 766 ensuring a thorough mixing and hypochlorous acid concentration as it exists at the bottom. The pressure pump 755 pressurizes the fluid sprayed through the atomizing nozzle 735. As the fluid passes into the nozzle, an electrostatic charge is induced into the atomized spray at the charged annular element 730. Controller 750 can adjust the concentration of HOCL by adjusting the voltage applied to produce the desired concentration required selected by the user on the touch screen display 740. During use and LED light 760 provides the user with task lighting. The system is housed in a durable plastic housing 720 that allows the unit to be held and operated with one hand. The controller and sensing methods ensure consistent production of hypochlorous acid. PH and salinity sensors are incorporated to provide feedback to the controller for consistency in hypochlorous production based on the selected concentration on touch screen display 740.

The system described above can be used to generate other cleaning solutions other than a hypochlorous acid solution by using a different feeder solution. For example, in an alternative embodiment, the cleaning solution generated by the system is a degreasing solution produced by using a feeder solution that comprises a mixture of water and potassium carbonate therein. The system in this instance is operated identically to the exemplary embodiments described above with the exception of the feeder solution and the resulting cleaning solution produced.

Since various modifications can be made in the invention as herein above described, and many apparently widely different embodiments of same made, it is intended that all matter contained in the accompanying specification shall be interpreted as illustrative only and not in a limiting sense.

1. A portable hypochlorous acid generating device comprising:
a supply tank arranged to contain a feeder solution comprising a mixture of water, sodium chloride and acetic acid therein;
an electrolytic cell comprising (i) a chamber, (ii) an inlet in communication between the chamber and the supply tank so as to be arranged to receive the feeder solution through the inlet into the chamber from the supply tank, (iii) two or more electrodes supported within the chamber so as to be arranged to produce a hypochlorous acid solution from the feeder solution by electrolysis, and (iv) a liquid outlet arranged to discharge the hypochlorous acid solution from the chamber through the liquid outlet;
a pump in communication with the liquid outlet of the electrolytic cell to produce a pressurized flow of the hypochlorous acid solution;
an atomizing nozzle in communication with the pump so as to be arranged to receive the pressurized flow of the hypochlorous acid solution and discharge the pressurized flow of the hypochlorous acid solution as an atomized mist; and
a controller arranged to actuate the electrodes of the electrolytic cell to produce the hypochlorous acid solution in the chamber when the pump is actuated to discharge the atomized mist from the atomizing nozzle.

2. The device according to claim 1 further comprising a trigger switch operatively connected to the controller, the controller being arranged to operate the pump and the electrodes of the electrolytic cell together in response to actuation of the trigger switch.

3. The device according to claim 1 further comprising a user input in communication with the controller so as to be arranged to receive a selected hypochlorous acid concentration input from a user, wherein the controller is arranged to adjust voltage applied to the electrodes of the electrolytic cell in response the hypochlorous acid concentration input received by the controller whereby a concentration of hypochlorous acid within the hypochlorous acid solution can be adjusted.

4. The device according to claim 1 further comprising a feeder solution sensor in communication with the controller, wherein the feeder solution sensor is arranged to measure electrical conductivity of the feeder solution in the supply tank, and wherein the controller is arranged to adjust voltage applied to the electrodes of the electrolytic cell in response to the measured electrical conductivity so as to maintain a consistent output of the hypochlorous acid solution from the chamber.

5. The device according to claim 1 wherein the supply tank is supported at least partly above the electrolytic cell, and wherein the inlet of the chamber is arranged to receive the feeder solution from the supply tank under force of gravity alone.

6. The device according to claim 1 wherein the electrolytic cell further comprises a gas outlet at a top end of the chamber, the gas outlet being arranged to discharge gas from the chamber through the gas outlet, the gas outlet including a discharge valve arranged to prevent discharge of fluid therethrough in response to a level of the hypochlorous acid solution in the chamber being at or above a prescribed upper limit.

7. The device according to claim 6 wherein the discharge valve comprises a float movable with an operating level of the hypochlorous acid solution in the chamber and wherein the discharge valve is arranged to close in response to the float being displaced by the operative level of the hypochlorous acid solution up to said prescribed upper limit.

8. The device according to claim 1 wherein the inlet is located at a top of the chamber, wherein the liquid outlet is located at a bottom of the chamber, and wherein the electrolytic cell further comprises baffles supported within the chamber between the inlet above and the liquid outlet below.

9. The device according to claim 8 wherein the electrodes comprise first and second electrode plates mounted parallel to one another within the chamber such that inner side surfaces of the electrode plates define a prescribed electrode gap therebetween and an opposing outer side surface of each electrode plate faces outwardly away from the other electrode plate, and wherein the baffles comprise insulated members mounted adjacent the outer side surface of at least one of the electrode plates.

10. The device according to claim 9 wherein the baffles extend upwardly and inwardly from respective opposing side edges of said at least one of the electrode plates towards an upright flow path extending along said at least one of the electrode plates at an intermediate location between the opposing side edges whereby a flow of gasses produced on said at least one of the electrode plates is directly upwardly and inwardly towards the upright flow path by the baffles.

11. The device according to claim 1 wherein the baffles protrude perpendicularly outwardly from the outer side surface of said at least one of the electrode plates.

12. The device according to claim 1 wherein the electrode plates comprise a perforated sheet material, and wherein the baffles are formed of an insulating material abutted against the outer side surface of said at least one of the electrode plates.

13. The device according to claim 1 wherein each baffle occupies a full depth protruding perpendicularly outward from the outer side surface of said at least one of the electrode plates to a corresponding boundary wall of the chamber.

14. The device according to claim 1 wherein the pump is mounted adjacent to the electrolytic cell, and wherein the pump and the electrolytic cell are connected by a vibration transmitting structure whereby vibration from the pump assists in releasing gas bubbles from the electrodes to improve electrode to fluid contact efficiency.

15. The device according to claim 1 further comprising:
a conductive member supported in proximity the atomizing nozzle; and
a transformer operatively connected to the conductive member so as to be arranged to charge the conductive member and transfer electric charge to the atomized mist discharged by the atomizing nozzle;
wherein the transformer is supported adjacent to the atomizing nozzle and spaced from the controller.

16. The device according to claim 15 further comprising
an air duct having a duct outlet adjacent to the atomizing nozzle, the air duct receiving the transformer therein;
a fan in communication with the direct so as to be arranged to direct a flow of air through the air duct, about the transformer, and out of the duct outlet in a common flow direction with the atomized mist of the hypochlorous acid solution discharged by the atomizing nozzle whereby the flow of air increases travel distance of the atomized mist and increases electrostatic charge imparted to the mist.

17. The device according to claim 15 further comprising (i) a main housing supporting the supply tank, the electrolytic cell and the pump thereon, (ii) a handheld housing supporting the atomizing nozzle and the transformer thereon, and (iii) a flexible line tethering the handheld housing to the main housing, the flexible line being in communication between the pump and the atomizing nozzle to communicate the pressurized flow of the hypochlorous acid solution from the pump to the atomizing nozzle therethrough.

18. The device according to claim 1 further comprising a pH sensor operatively connected to the chamber so as to measure a pH value of the hypochlorous acid solution in the chamber, wherein the controller is arranged to cease operation of the pump and actuation of the electrodes of the electrolytic cell in response to the pH value measured by the pH sensor meeting a pH limit stored on the controller.

19. The device according to claim 1 wherein the controller is arranged to cease operation of the pump and actuation of the electrodes of the electrolytic cell in response to voltage and current applied to the electrolytic cell by the controller meeting a prescribed limit stored on the controller.

20. The device according to claim 1 further comprising a battery providing electrical power to the electrolytic cell and the pump, wherein the battery, the electrolytic cell, the pump, the controller, and the supply tank being commonly supported on a main housing which is portable and arranged to be carried by a single operator.

21. The device according to claim 1 wherein:
the electrolytic cell is supported on a main housing;
the supply tank is movable from a mounted position on the main housing to a released position separated from the main housing; and
the supply tank includes a coupling valve supported on an outlet of the supply tank in which the coupling valve is biased to a closed position when the supply tank is in the released position and in which the coupling valve is arranged to be displaced to an open position to allow communication of the feeder solution to the chamber of the electrolytic cell responsive to mounting of the supply tank onto the main housing in the mounted position.

22. A portable cleaning solution generating device comprising:
a supply tank arranged to contain a feeder solution;
an electrolytic cell comprising (i) a chamber, (ii) an inlet in communication between the chamber and the supply tank so as to be arranged to receive the feeder solution through the inlet into the chamber from the supply tank, (iii) two or more electrodes supported within the chamber so as to be arranged to produce the cleaning solution from the feeder solution by electrolysis, and (iv) a liquid outlet arranged to discharge the cleaning solution from the chamber through the liquid outlet;
a pump in communication with the liquid outlet of the electrolytic cell to produce a pressurized flow of the cleaning solution;
an atomizing nozzle in communication with the pump so as to be arranged to receive the pressurized flow of the cleaning solution and discharge the pressurized flow of the cleaning solution as an atomized mist; and
a controller arranged to actuate the electrodes of the electrolytic cell to produce the cleaning solution in the chamber when the pump is actuated to discharge the atomized mist from the atomizing nozzle.

23. The device according to claim 22 wherein the feeder solution comprises a mixture of water, sodium chloride and acetic acid therein, and wherein the electrolytic cell is arranged to produce the cleaning solution from the feeder solution by electrolysis such that the cleaning solution comprises a hypochlorous acid solution.

24. The device according to claim 22 wherein the feeder solution comprises a mixture of water and potassium carbonate therein, and wherein the electrolytic cell is arranged to produce the cleaning solution from the feeder solution by electrolysis such that the cleaning solution comprises a degreasing solution.
